(19) United States
(12) Patent Application Publication
(10) Pub. No.: US 2021/0024542 A1
(43) Pub. Date: Jan. 28, 2021

(54) RAPAMYCIN ANALOG FOR PREVENTION AND/OR TREATMENT OF CANCER

(71) Applicant: Torcept Therapeutics, Inc., Middlebury, VT (US)

(72) Inventors: Timothy S Powers, Sausalito, CA (US); Bryan F Cox, Spring Grove, IL (US); Richard B Marshak, Cornwall, VT (US)

(73) Assignee: Torcept Therapeutics, Inc., Middlebury, VT (US)

(21) Appl. No.: 16/968,207

(22) PCT Filed: Feb. 8, 2019
(86) PCT No.: PCT/US2019/017327

§ 371 Date: Aug. 7, 2020

Related U.S. Application Data
(60) Provisional application No. 62/628,520, filed on Feb. 9, 2018.

(51) Int. Cl. C07D 498/18; A61P 35/00

(52) U.S. Cl. CPC C07D 498/18; A61K 45/06; A61P 35/00

(57) ABSTRACT

The present invention relates to a novel rapamycin analogue, mixtures, methods for its production, and its use in cancer therapy (e.g., prevention and/or treatment).

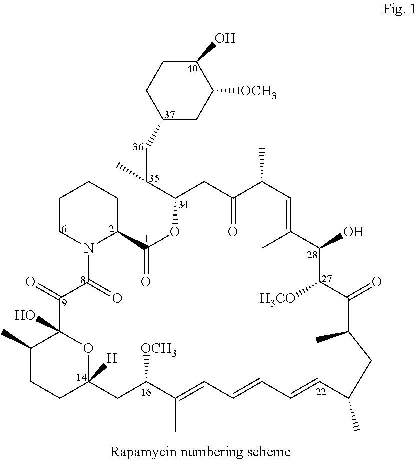 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

### RELATED APPLICATIONS

This application claims priority to U.S. Ser. No. 62/628,520 filed on Feb. 9, 2018.

### BACKGROUND OF THE DISCLOSURE

Rapamycin (sirolimus) is a polyketide that is used to coat coronary stents and prevent organ transplant rejection. The art also suggests that rapamycin and rapamycin analogs can be used to treat lymphangioleiomyomatosis, pulmonary inflammation (U.S. Pat. No. 5,080,999), insulin dependent diabetes (U.S. Pat. No. 5,362,718 citing Fifth Int. Conf. Inflamm. Res. Assoc. 121 (Abstract), (1990)), certain coronary diseases (Morris, (1992) Heart Lung Transplant 11:197), leukemia and lymphoma (European Patent Application 0 525 960), and ocular inflammation (European Patent Application 0 532 862).

Rapamycin is produced by Streptomyces hygroscopicus NRRL 5491 (Sehgal et al., 1975; Vezina et al., 1975; U.S. Pat. Nos. 3,929,992; 3,993,749). For the purpose of this disclosure, rapamycin is described by the numbering convention of McAlpine et al. (1991) (FIG. 1) in preference to the numbering conventions of Findlay et al. (1980) or Chemical Abstracts (11th Cumulative Index, 1982-1986 p60719CS).

U.S. Pat. No. 5,362,718 discloses acylated prodrugs of rapamycin.

Rapamycin and its commercially available analogs Temsirolimus and Everolimus inhibit activation of T cells and B cells by binding to mTOR which, among other things, reduces the production of interleukin-2. mTOR is the catalytic subunit of two structurally distinct complexes: mTORC1 and mTORC2 (Wang et al. (2006) Journal of Biological Chemistry, 281: 24293-303). mTORC1 and mTORC2 localize to different subcellular compartments, which affects their activation and function.

Scientific evidence suggests that mTORC1 functions as a sensor of cellular nutritional and energy status and has a role in the regulation of protein synthesis (Hay et al. (2004) Genes & Development 18: 1926-45; Kim et al. (2002) Cell, 110: 163-75). The activity of mTORC1 is regulated by rapamycin analogs, insulin, growth factors, phosphatidic acid, some amino acids and amino acid derivatives, mechanical stimuli, and oxidative stress.

Scientific evidence suggests that mTORC2 functions an important regulator of the actin cytoskeleton through its stimulation of F-actin stress fibers, paxillin, RhoA, Rac1, Cdc42, and protein kinase Cα (Sarbassov et al. (2004) Current Biology 14:1296-302). mTORC2 also affecting metabolism and survival apparently through phosphorylation of Akt/PKB (Betz et al. (2013) PNAS 110:12526-34). Akt phosphorylation by mTORC2 interacts with PDK1 and leads to full Akt activation (Sarbassov et al. (2005) Science 307:1098-101; Stephens et al. (1998) Science 279: 710-4). In addition, mTORC2 is capable of activating IGF-IR and InsR (Yin et al. (2016) Cell Research 26:46-65).

While not intending to be bound be theory, we believe that rapamycin-like polyketide inhibitors of mTOR having a more balanced (e.g., less selective) ability to inhibit mTORC1 and mTORC2 are preferred for the treatment of certain cancers because inhibition of both mTORC1 and mTORC2 disables an escape mechanism through which drug resistance can develop.

Rapamycin analogs (including Rapamycin) have significant therapeutic value (Huang et al, 2003). These polyketides are a potent inhibitor of the mammalian target of rapamycin (mTOR), a serine-threonine kinase downstream of the phosphatidylinositol 3-kinase (PI3K)/Akt (protein kinase B) signaling pathway that mediates cell survival and proliferation. This inhibitory activity is gained after rapamycin binds to the immunophilin FK506 binding protein 12 (FKBP12) (Dumont, F. J. and Q. X. Su, 1995). In T cells, rapamycin inhibits signaling from the IL-2 receptor and subsequent autoproliferation of the T cells resulting in immunosuppression. Rapamycin is marketed as an immunosuppressant for the treatment of organ transplant patients to prevent graft rejection (Huang et al, 2003). In addition to immunosuppression, rapamycin has found therapeutic application in cancer (Vignot et al, 2005).

Rapamycin and many rapamycin analogs have disadvantages including inducement of hyperlipidemia, cellular efflux mediated by P-glycoprotein (“P-gp”; LaPlante et al, 2002, Crowe et al, 1999) and other efflux mechanisms which pumps the compound out of cells and tends to decrease effectiveness of administered drug compound and presents challenges to the treatment of multidrug resistant cancer. Hepatic first pass loss of rapamycin is also high, which contributes further to its low oral bioavailability. The low oral bioavailability of rapamycin causes significant inter-individual variability resulting in inconsistent therapeutic outcome and difficulty in clinical management (Kuhn et al, 2001, Crowe et al, 1999).

A wide range of synthesized rapamycin analogues using the chemically available sites of the molecule are known in the art. Chemically available sites on the molecule for derivatization or replacement are known in the art to include, for example, C40 and C28 hydroxyl groups (e.g. U.S. Pat. Nos. 5,665,772; 5,362,718); C39 and C16 methoxy groups (e.g. WO 96/41807; U.S. Pat. No. 5,728,710); C32, C26 and C9 keto groups (e.g. U.S. Pat. Nos. 5,378,836; 5,138,051; 5,665,772); hydrogenation at C17, C19 and/or C21 (e.g. U.S. Pat. Nos. 5,391,730; 5,023,262); and/or the formation of oximes at C32, C40 and/or C28, (e.g., U.S. Pat. Nos. 5,563,145, 5,446,048). Analogues exhibiting resistance to metabolic attack (e.g. U.S. Pat. No. 5,912,253); bioavailability (e.g. U.S. Pat. Nos. 5,221,670; 5,955,457; WO 98/04279); and/or the production of prodrugs (e.g. U.S. Pat. Nos. 6,015,815; 5,432,183) have also been developed. Thus, it is understood in the art that the number of pharmaceutically useful and interesting analogs of rapamycin is very high and difficult to quantify.

### SUMMARY OF THE DISCLOSURE

In some embodiments, this disclosure provides a polyketide similar to rapamycin that has an unexpected and beneficial pharmaceutical uses in preventing and/or treating cancer. In some embodiments, this disclosure provides compositions comprising the polyketide of Formula I, and other polyketides. In some embodiments, methods of preventing and/or treating cancer in a mammal comprising administering the compound of Formula I, and/or compositions and/or mixtures comprising the same are provided. Other embodiments are also contemplated as described herein and/or as may be ascertained by those of ordinary skill in the art.

### BRIEF DESCRIPTION OF THE DRAWINGS

FIG. 1. The rapamycin numbering schema used in this document.

FIG. 2. Dosing of Formula I at 2 mg/kg or 10 mg/kg IP (QDx3 days) in Mouse Whole Blood.

FIG. 3. Tumor volume over time following administration of Formula I. FIG. 3A: Mean Tumor Volume Over Time. FIG. 3B: Median Tumor Volume Over Time; FIG. 3C: Mean Percent Tumor Volume Over Time. FIG. 3D: Median Percent Tumor Volume Over Time.

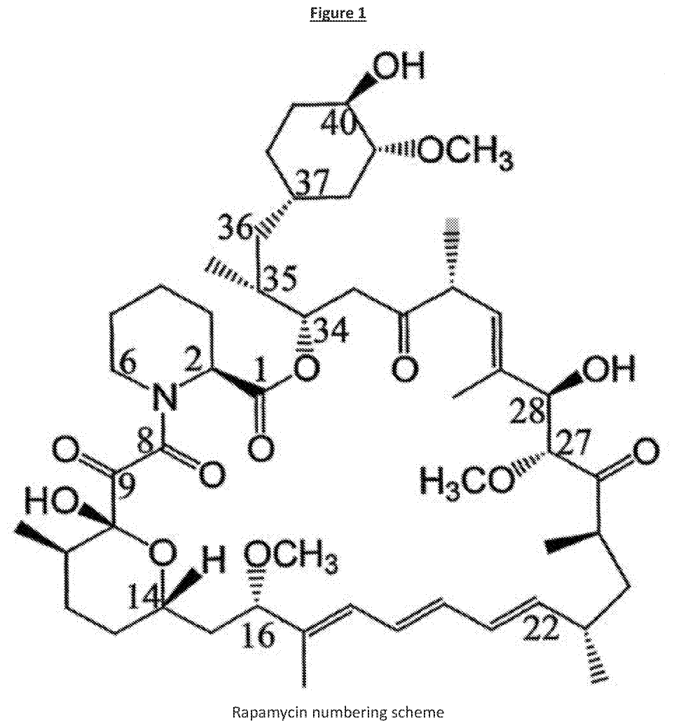

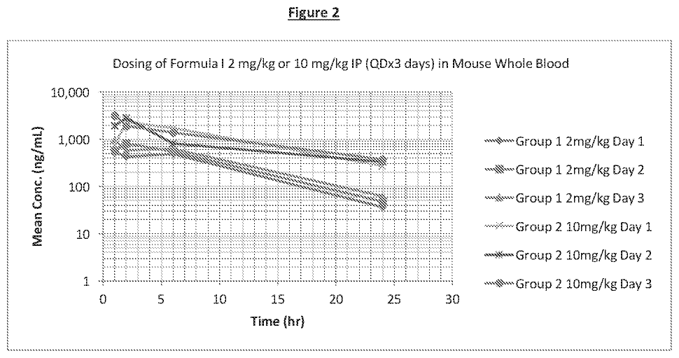

### DETAILED DESCRIPTION

This disclosure provides the polyketides of Formula I (37R-hydroxynorbornylrapamycin), compositions comprising the same, and the use of the same in preventing and/or treating cancer. Compounds of Formula I have surprising and unexpectedly beneficial properties for the prevention and/or treatment of cancer. The compound of Formula I is shown below:

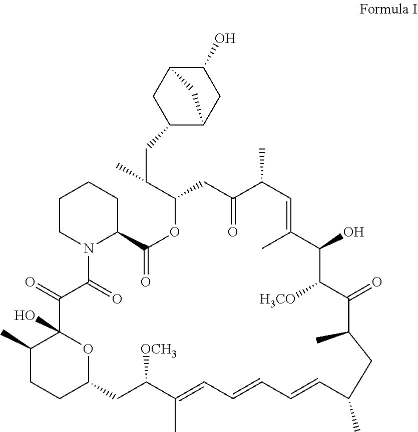 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2C[C@@H]3C[C@H]2C[C@H]3O)CC(=O)[C@H](C)/C=C(\C)[C@@H](O)[C@@H](OC)C(=O)[C@H](C)C[C@H](C)/C=C/C=C/C=C/1C]

The compound of Formula I (37R-hydroxynorbornylrapamycin) is also described in U.S. Pat. No. 9,382,266 B2, which is hereby incorporated by reference in its entirety into this disclosure.

Compositions comprising the compound of Formula I are optionally, but need not be, pure. The polyketide of Formula I can be present in mixtures in which essentially all of the polyketide in the mixture is the polyketide of Formula I, in which 99.9% by weight of the polyketide in the mixture is the polyketide of Formula I, in which 99.5% of the polyketide in the mixture is the polyketide of Formula I, in which at least 99% of the polyketide in the mixture is the polyketide of Formula I, in which 98% of the polyketide in the mixture is the polyketide of Formula I in which at least 95% of the polyketide in the mixture is the polyketide of Formula I, in which at least 90% of the polyketide in the mixture is the polyketide of Formula I, in which 80% of the polyketide in the mixture is the polyketide of Formula I, or in which the 70% of the polyketide in the mixture is the polyketide of Formula I.

Furthermore, in each of the foregoing aspects of the present invention the compound of Formula I is optionally provided as a salt, a solvate, or an ester of the compound of Formula I. Pharmaceutically acceptable salts of the polyketide of Formula I include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmitic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, optionally can be useful in the preparation of salts useful as intermediates in obtaining the compound of Formula I and its pharmaceutically acceptable salts. More specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine and procaine salts. In an aspect of the present invention, pharmaceutically acceptable salts of the polyketide of Formula I are combined together with one or more pharmaceutically acceptable excipients, diluents, or carriers.

Similarly, the polyketides of Formula I, and pharmaceutically acceptable salts thereof, optionally can be solvates, including alcoholic solvates and hydrates.

The polyketide of Formula I can be provided in a pure form for example in a crystalline or powdered form or diluted in at least one pharmaceutically acceptable buffer, carrier, or excipient. Pharmaceutically acceptable buffers, carriers and excipients in the context of the present invention preferably do not adversely interact with the polyketide of Formula I, provide for stable formulations for suitable time periods, and are not unduly deleterious to most recipients thereof.

In some embodiments, solutions or suspensions of a compound of Formula I also contain excipients such as, e.g., N,N-dimethylacetamide, dispersants e.g. polysorbate 80, surfactants, and solubilizers, e.g. polyethylene glycol, and/or Phosal 50 PG (which consists of phosphatidylcholine, soya-fatty acids, ethanol, mono/diglycerides, propylene glycol and ascorbyl palmitate).

Compositions comprising the compound of Formula I can be administered via any suitable route or means including, but not limited to, parenterally, orally, topically (including buccal, sublingual, or transdermally), via a medical device such as a stent, by inhalation, or via injection (e.g., subcutaneously, intramuscularly, or intravenously). The treatment optionally consists of a single dose, but preferably in many embodiments is a multiplicity of administrations over time. The skilled artisan will recognize that the optimal quantity and spacing of individual dosages of a compound of Formula I will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

In some embodiments, the compound of Formula I, or a composition comprising the same, is administered as the sole active pharmaceutical agent. Thus, in some embodiments, the compound of Formula I, or a composition comprising the same, is administered to a mammal such as a human being to prevent and/or treat a disease as a pharmaceutical composition that optionally contains one or more pharmaceutical excipients, but no other active agent(s). In some embodiments, the compound of Formula I, or a composition comprising the same, is administered to a mammal such as a human being to prevent and/or treat a disease as a pharmaceutical composition comprising at least one other active agent, and optionally also contains one or more pharmaceutical excipients. In some embodiments, the compound of Formula I, or a composition comprising the same, is administered to a mammal such as a human being to prevent and/or treat cancer as a pharmaceutical composition optionally comprising at least one other active agent, and optionally also containing one or more pharmaceutical excipients, optionally with at least one other composition also comprising at least one other active agent and optionally also containing one or more pharmaceutical excipients (e.g., two compositions, one containing at least the compound of Formula I, and the other composition comprising at least one other active agent). Multiple compositions, each comprising one or more active agents (one of such compositions comprising the compound of Formula I), may also be administered to prevent and/or treat cancer. Such active agents and/or compositions may be administered simultaneously or sequentially, or some combination thereof, and may be administered at the same or different sites on the mammal, and/or through the same or different routes of administration.

Active agents that may be administered to a mammal in order to prevent and/or treat disease along with the compound of Formula I, or a composition comprising the same, either simultaneously in administration or as a single composition, or separately, include but are not limited to one or more chemotherapeutic agents, anti-cancer agents, radiation therapy, immune modulators, such as, for instance, and without limitation, one or more of any of: an anti-cancer agent reduces or minimizes any undesired side-effects associated with certain types of cancer treatment (e.g., fatigue, anemia, appetite changes, bleeding problems, diarrhea, constipation, hair loss, nausea, vomiting, pain, peripheral neuropathy, swelling, skin and nail changes, urinary and bladder changes, trouble swallowing, etc.), alkylating agents (e.g., nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, sodium estramustine phosphate, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin, and the like), antimetabolites (e.g., mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emitefur, and the like), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine and the like), anticancer antibiotics (e.g., actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, and the like), plant-derived anticancer agents (e.g., etoposide, etoposide phosphate, vinblastine sulfate, vincristine sulfate, vindesine sulfate, teniposide, paclitaxel, docetaxel, vinorelbine, and the like), immunotherapeutic agents (e.g., antibodies (e.g., anti-PD1 antibodies, PD-L1 antibodies, anti-CTLA4 antibodies, anti-CD20 antibodies, anti-CD25 antibodies, HER2 antibody (e.g., trastuzumab), imatinib mesylate, ZD1839 or EGFR antibody (e.g., cetuximab), antibody to VEGF (e.g., bevacizumab), VEGFR antibody, VEGFR inhibitor, and EGFR inhibitor (e.g., erlotinib)), picibanil, krestin, sizofuran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole, and the like), methotrexate, doxorubicin, 5-fluorouracil, vincristine, vinblastine, pamidronate disodium, anastrozole, exemestane, cyclophosphamide, epirubicin, toremifene, letrozole, trastuzumab, megestrol, tamoxifen, paclitaxel, docetaxel, capecitabine, goserelin acetate, a glycolytic inhibitor; one or more hormonal therapeutic agents (fosfestrol, diethylstylbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, Tadenan, mepartricin, raloxifene, ormeloxifene, levormeloxifene, anti-estrogens (e.g., tamoxifen citrate, toremifene citrate, and the like), ER down-regulator (e.g., fulvestrant and the like), human menopausal gonadotrophin, follicle stimulating hormone, pill preparations, mepitiostane, testroactone, aminoglutethimide, LH-RH agonists (e.g., goserelin acetate, buserelin, leuprorelin, and the like), drooxifene, epitiostanol, ethinylestradiol sulfonate, aromatase inhibitors (e.g., fadrozole hydrochloride, anastrozole, retrozole, exemestane, vorozole, formestane, and the like), anti-androgens (e.g., flutamide, bicartamide, nilutamide, and the like), 5α-reductase inhibitors (e.g., finasteride, dutasteride, epristeride, and the like), adrenocorticohormone drugs (e.g., dexamethasone, prednisolone, betamethasone, triamcinolone, and the like); one or more androgen synthesis inhibitors (e.g., abiraterone, and the like); one or more retinoids and/or drugs that retard retinoid metabolism (e.g., liarozole, and the like), etc. and LH-RH agonists (e.g., goserelin acetate, buserelin, leuprorelin)); L-asparaginase, aceglatone, procarbazine hydrochloride, protoporphyrin-cobalt complex salt, mercuric hematoporphyrin-sodium; topoisomerase I inhibitors (e.g., irinotecan, topotecan, and the like), topoisomerase II inhibitors (e.g., sobuzoxane, and the like), differentiation inducers (e.g., retinoid, vitamin D, and the like), α-blockers (e.g., tamsulosin hydrochloride, naftopidil, urapidil, alfuzosin, terazosin, prazosin, silodosin, and the like), serine/threonine kinase inhibitors, endothelin receptor antagonists (e.g., atrasentan, and the like), proteasome inhibitor (e.g., bortezomib, and the like), Hsp 90 inhibitors (e.g., 17-AAG, and the like), spironolactone, minoxidil, 11α-hydroxyprogesterone, bone resorption inhibiting/metastasis suppressing agents (e.g., zoledronic acid, alendronic acid, pamidronic acid, etidronic acid, ibandronic acid, clodronic acid), angiogenesis inhibitors (e.g., nintedanib (BIBF 1120, Vargatef), bevacizumab (Avastin), everolimus (Afinitor), temsirolimus (Torisel), lenalidomide (Revlimid), pazopanib (Votrient), ramucirumab (Cyramza), sorafenib (Nexavar), sunitinib (Sutent), thalidomide (Thalomid), vandetanib (Caprelsa), cediranib (Recentin), axitinib (Inlyta), motesanib, vatalanib, dovitinib, brivanib, linifanib, tivozanib, lenvatinib, regorafenib (Stivarga), foretinib, telatinib, cabozantinib (Cometriq), nilotinib (Tasigna), tandutinib, imatinib (Gleevec), BMS-690514, quizartinib (AC220), orantinib, olaratumab, erlotinib (Tarceva), gefitinib (Iressa), afatinib (Gilotrif), lapatinib (Tykerb), varlitinib, AEE-788, trastuzumab (Herceptin), cetuximab (Erbitux), panitumumab (Vectibix), nimotuzumab, pertuzumab (Omnitarg), ertumaxomab, or zalutumumab. In some embodiments, the angiogenesis inhibitor is nintedanib (BIBF 1120), everolimus (Afinitor), temsirolimus (Torisel), pazopanib (Votrient), axitinib (Inlyta), bevacizumab (Avastin), sorafenib (Nexavar), sunitinib (Sutent), thalidomide (Thalomid), dovitinib, regorafenib (Stivarga), or imatinib (Gleevec)), and the like, and/or combinations and/or mixtures thereof, optionally along with any other active agents described herein or that may be otherwise available to those of skill in the art. The compound of Formula I, or a composition comprising the same, with or without at least one other active agent, may also be administered in conjunction with any one or more of surgery, radiotherapy, gene therapy, thermotherapy, cryotherapy, laser cauterization, and the like, and/or any combinations thereof, optionally along with any of the active agents described herein or that may be otherwise available to those of skill in the art.

Tablets containing the compound of Formula I, or a composition comprising the same, optionally contain excipients such as microcrystalline cellulose, lactose (e.g., lactose monohydrate or lactose anhydrous), sodium citrate, calcium carbonate, dibasic calcium phosphate and glycine, butylated hydroxytoluene (E321), crospovidone, hypromellose, disintegrants such as starch (preferably corn, potato or tapioca starch), sodium starch glycollate, croscarmellose sodium, and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, and talc are optionally included.

Solid compositions of a similar type can also be employed as fillers in gelatin capsules. Preferred excipients in this regard include lactose, starch, a cellulose, milk sugar or high molecular weight polyethylene glycols. For aqueous suspensions and/or elixirs, the compounds of Formula I can be combined with various sweetening or flavoring agents, coloring matter or dyes, with emulsifying and/or suspending agents and with diluents such as water, ethanol, propylene glycol and glycerin, and combinations thereof.

A tablet can be made by compression or molding, optionally with one or more accessory ingredients. Compressed tablets can be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Molded tablets can be made by molding in a suitable machine a mixture of the powdered compound moistened with an inert liquid diluent. The tablets can optionally be coated or scored and can be formulated so as to provide slow or controlled release of the active ingredient therein using, for example, hydroxypropylmethylcellulose in varying proportions to provide desired release profile.

For convenience, the formulations are optionally presented in unit dosage form and can be prepared by any of the methods well known in the art of pharmacy. Such methods include the step of bringing into association the active ingredient (compound of Formula I) with the carrier which constitutes one or more accessory ingredients. In general, the formulations are prepared by uniformly and intimately bringing into association the active ingredient with liquid carriers or finely divided solid carriers or both, and then, if necessary, shaping the product.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oeyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions, the active ingredient can be dissolved in water for injection and filter sterilized before filling into a suitable vial or ampoule and sealing.

Advantageously, agents such as local anesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilized by exposure to ethylene oxide before suspending in the sterile vehicle. Advantageously, a surfactant or wetting agent is included in the composition to facilitate uniform distribution of the active ingredient.

The compound of Formula I, or a composition comprising the same, may also be administered using medical devices known in the art. For example, in one embodiment, a pharmaceutical composition described herein can be administered with a needleless hypodermic injection device, such as the devices disclosed in U.S. Pat. No. 5,399,163; 5,383,851; 5,312,335; 5,064,413; 4,941,880; 4,790,824; or 4,596,556. Useful examples of well-known implants and modules include but are not limited to U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. In a specific embodiment, the polyketides (e.g., of Formula I or II) and compositions comprising the same may be administered using a drug-eluting stent, for example, such as one corresponding to those described in WO 01/87263 and related publications or those described by Perin (Perin, E C, 2005). Many other such implants, delivery systems, and modules are known to those skilled in the art.

The compound of Formula I, or a composition comprising the same, can be administered to treat, prevent, or mitigate cancer or a cancer-related medical condition in a mammal in need thereof. In a preferred embodiment, the mammal is a human. Any appropriate medical condition of the mammal can be treated by administering a pharmaceutically-appropriate quantity (e.g., a therapeutically effective amount) of the compound of Formula I to a mammal in need thereof. An ordinarily skill artisan can readily select the route of administration of the compound of Formula I, or a composition comprising the same, as well as the quantity following routine studies, guidelines and procedures. The dosage to be administered of the compound of Formula I will vary according to the particular compound, the type of cancer involved, the subject, and the nature and severity of the disease and the physical condition of the subject, and the selected route of administration. The appropriate dosage can be readily determined by a person skilled in the art. For example, without limitation, a dose of about 0.1 mg up to 100 mg daily, and optionally about 0.1 to 15 mg daily (or a higher dose given less frequently) can be administered. In some embodiments, a dose of about 1 or 3 mg/kg of Formula I i.v. or 1, 5 or 10 mg/kg of the compound of Formula I p.o., or a dose of from 1-20 mg/kg (e.g., about any of 1, 2, 3, 4, 5, 6, 7, 8, 9, 10, 11, 12, 13, 14, 15, 17, 18, 19 or 20 mg/kg) can be administered one or more times.

The compositions may contain any suitable combination of the compound of Formula I, or a composition comprising the same, and other components. In some preferred embodiments, the compositions of the invention contain from 0.1 weight % to 70 weight % of the compound of Formula I, preferably from 5-60 weight %, more preferably from 10 to 30 weight %, of the compound of Formula I, depending on the method of administration and other factors.

The compounds of Formula I can be purified by any suitable separation technology including, but not limited to, preparative-scale chromatography.

Thus, in some embodiments, this disclosure provides a compound of Formula I as described above, and/or a pharmaceutically acceptable salt, solvate, ester, mixture thereof, and/or composition comprising the same and/or one or more pharmaceutically acceptable carriers for use in preventing and/or treating cancer. In some embodiments, the drug is a polyketide of Formula I, as well as pharmaceutically acceptable salts, solvates, and hydrates of the compound of Formula I. In some embodiments, this disclosure provides a composition comprising about 70% or more, about 80% or more, about 90% or more (i.e., “substantially pure”), about 95% or more, or about 99% or more of the compound of Formula I, a pharmaceutically acceptable salt thereof, a solvate thereof, an ester thereof, and/or mixtures of the foregoing for use in preventing and/or treating cancer. In some embodiments, the composition contains an essentially pure mixture, wherein an essentially pure mixture may contain trace amounts or pharmaceutically insignificant amounts of other polyketides, of a compound selected from the group consisting of the compound of Formula I, pharmaceutically acceptable salts, solvates, and esters thereof and mixtures of the foregoing. In some embodiments, this disclosure provides a pharmaceutical composition comprising a pharmaceutically acceptable carrier, diluent or excipient and the compound of Formula I, wherein the pharmaceutically acceptable salt, solvate, and/or hydrate of the compound of Formula I comprises at least about 70%, at least about 80%, at least about 90%, at least about 95%, or at least about 98% of the polyketide component of the pharmaceutical composition. In some embodiments of such compositions, the compound of Formula I is essentially the only polyketide in the pharmaceutical composition. In some embodiments, the solvate, if present, is a hydrate.

In some embodiments, methods for inhibiting the proliferation of a cell by contacting said cell with an antiproliferative amount of a compound of Formula I, pharmaceutically acceptable salt thereof, solvate thereof, ester thereof, or mixture thereof and/or comprising Formula I; and/or a composition comprising Formula I. In some embodiments, the cell is human cell such as, preferably a human cancer cell (such as but not limited to, e.g., adenocarcinoma, bladder cancer, blood cancer, bone cancer, brain cancer, solid tumor, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and/or transitional cell carcinoma). In some embodiments, the method is an in vitro method or an in vivo method. In some embodiments, the antiproliferative or anticancer effect of the compound of Formula I is determined using the cell count EC₅₀, the IC₅₀, and/or GI₅₀. In some exemplary embodiments, the EC₅₀, the IC₅₀, and/or GI₅₀ is about or between any of the values shown in Table 2, and/or in some embodiments at least about 10% that of rapamycin. For instance, in some embodiments, the EC50 (μM) can be from about 10⁻⁷ (e.g., about 3.99E-06 (3.99×10⁻⁶)) to 1 μM, including any value in between; the IC50 (μM) can be from about 10⁻⁵ (e.g., about 9.56E-04 (9.56×10⁻⁴)) to 1 μM, including any value in between; and, the GI50 (μM) can be from about 10⁻⁵ (e.g., about 9.99E-04 (9.99×10⁻⁴)) to 1 μM, including any value in between. Other EC₅₀, the IC₅₀, and/or GI₅₀ values are also contemplated as would be understood by those of ordinary skill in the art.

In some embodiments, this disclosure also provides methods for treating a mammal in need of prevention and/or treatment of cancer, the method comprising administering to said mammal (e.g., a human being) an effective amount (e.g., a therapeutically effective amount) of the compound of Formula I, a pharmaceutically acceptable salt thereof, a solvate thereof, an ester thereof of the compound of Formula I, and/or a composition and/or mixture comprising the same. In some embodiments, the mammal has a disease selected from the group consisting of cancer (such as but not limited to, e.g., adenocarcinoma, bladder cancer, blood cancer, bone cancer, brain cancer, solid tumor, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and/or transitional cell carcinoma). In some embodiments, the method comprises administration of the compound of Formula I to a mammal at about 2 mg/kg or about 10 mg/kg to provide an approximate mean concentration in the whole blood of the mammal that provides a therapeutic effect (e.g., a therapeutically effective amount) for up to six hours following administration. In some embodiments, the method comprises administering the compound of Formula I to a mammal having a solid tumor and multiple administrations to the mammal are performed, and resulting in a reduction in the volume of the solid tumor (e.g., at least about any of 20%, 25%, 30%, 40%, 50%, or 60%). In some such embodiments, a significant reduction in tumor volume results from administration of a compound of Formula I for at least about eight consecutive days. In some embodiments, such as those using an animal model (e.g., a mouse), the compound of Formula I is administered to the mammal for 30 days and results mean differential tumor percent as compared to an untreated mammal with an adjusted P value of 0.0001 as determined by Dunnett's multiple comparison's test.

Any mode of administration may be utilized. In some embodiments, the compound, composition and/or mixture is administered by application to an implantable medical device (e.g., a stent).

In some embodiments, then, this disclosure provides methods for preventing and/or treating cancer in a mammal by administering a compound of Formula I, a pharmaceutically acceptable salt, solvate, ester, or mixture thereof in a therapeutically effective amount, and/or a composition comprising the same. In some embodiments, the cancer is selected from, but not limited to, one or more of a blood cancer, bone cancer, solid tumor, adenocarcinoma, brain cancer, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and transitional cell carcinoma. In some embodiments, the compound of Formula I, or compositions comprising the same, is administered as the sole active pharmaceutical agent. In some embodiments, the compound of Formula I, or a composition comprising the same, is administered in combination with one or more of a chemotherapeutic agent, anti-cancer agent, or immune modulator; and/or radiation therapy and/or surgery; and/or other treatment modality. In some embodiments, the administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately. In some embodiments, the compound of Formula I is provided as a bead, tablet, capsule, solution, solid, or suspension. Also provided are uses of a compound of Formula I in the preparation of a medicament for the prevention and/or treatment of cancer (e.g., as described above). In some embodiments, kits for preventing and/or treating cancer in which the kits comprise at least one therapeutically effective dose of the compound of Formula I, typically in a container, and instructions for preparing and/or using the at least one therapeutically effective dose for preventing and/or treating cancer using the same are also provided. In some embodiments, the instructions refer to administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately. In some embodiments, the kit provides the compound of Formula I as a bead, tablet, capsule, solution, or suspension. Other embodiments are also contemplated as would be understood by those of skill in the art.

When the terms treat, prevent, and/or ameliorate or derivatives thereof are used herein in connection with a given treatment for a given condition, it is meant to convey that the treated patient either does not develop a clinically observable level of the condition at all, or develops it more slowly and/or to a lesser degree than he/she would have absent the treatment. These terms are not limited solely to a situation in which the patient experiences no symptom of the condition whatsoever. For example, a treatment will be said to have prevented the condition if it is given during exposure of a patient to a stimulus that would have been expected to produce a given manifestation of the condition, and results in the patient's experiencing fewer and/or milder symptoms of the condition than otherwise expected. For instance, a treatment can “prevent” symptoms where the mammal expresses less symptoms than would have been observed in the absence of treatment; it does not imply that the mammal must not exhibit any symptoms. Similarly, reduce, reducing, and reduction as used herein in connection with prevention, treatment and/or amelioration of a given condition by a particular treatment typically refers to a subject developing an infection more slowly or to a lesser degree as compared to a control or basal level (e.g., of symptoms) in the absence of a treatment.

The terms “about”, “approximately”, and the like, when preceding a list of numerical values or range, refer to each individual value in the list or range independently as if each individual value in the list or range was immediately preceded by that term. The terms “about”, “approximately”, and the like mean that the values to which the same refer are exactly, close to, or similar thereto. As used herein, a subject or a host or a mammal is meant to be an individual. The subject can include mammals such as domesticated animals, such as cats and dogs, livestock (e.g., cattle, horses, pigs, sheep, and goats), laboratory animals (e.g., mice, rabbits, rats, guinea pigs) and birds. A mammal may also be a primate or a human. Optional or optionally means that the subsequently described event or circumstance can or cannot occur, and that the description includes instances where the event or circumstance occurs and instances where it does not. For example, the phrase “optionally the composition can comprise a combination” means that the composition may comprise a combination of different compounds and/or molecules, and/or may not include a combination such that the description includes both the combination and the absence of the combination (i.e., individual members of the combination). The term “combined” or “in combination” or “in conjunction” may refer to a physical combination of agents that are administered together or the use of two or more agents in a regimen (e.g., administered separately, physically and/or in time) for treating, preventing and/or ameliorating a particular disease. Ranges may be expressed herein as from about one particular value, and/or to about another particular value. When such a range is expressed, another aspect includes from the one particular value and/or to the other particular value. Ranges (e.g., 90-100%) are meant to include the range per se as well as each independent value within the range as if each value was individually listed. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Throughout the specification and the claims which follow, unless the context requires otherwise, the word ‘comprise’, and variations such as ‘comprises’ and ‘comprising’, will be understood to imply the inclusion of the stated subject matter (e.g., a composition), a stated integer, step, group of subject matter, integers or group of steps but not to the exclusion of any other subject matter, integer, step, group of integers or group of steps. All references referred to in this application, including patent and patent applications, are incorporated herein by reference into this disclosure in their entirety.

Certain embodiments are further described in the following examples. These embodiments are provided as examples only and are not intended to limit the scope of the claims in any way.

### EXAMPLES

### Example 1

This example compares the inhibition of proliferation the indicated cell lines, compared with that observed for staurosporin and rapamycin, using the OncoPanel™ cell proliferation assay which measures the proliferation response of cancer cell lines to drug treatments through high-content fluorescence imaging or bioluminescence.

Cells were grown in RPMI 1640, 10% FBS, 2 mM L-alanyl-L-glutamine, 1 mM Na pyruvate, or a special medium. Cells were seeded into 384-well plates and incubated in a humidified atmosphere of 5% CO₂ at 37° C. Compounds were added the day following cell seeding. At the same time, a time zero untreated cell plate was generated. After a 3-day incubation period, cells were fixed and stained to allow fluorescence imaging of nuclei. Compounds (1 mM stock solutions) were serially diluted in half-log steps from the highest test concentration (1 micromol), and assayed over 10 concentrations with a maximum assay concentration of 0.1% DMSO. Automated fluorescence microscopy was carried out using a Molecular Devices ImageXpress Micro XL high-content imager, and images were collected with a 4× objective. 16-bit TIFF images were acquired and analyzed with MetaXpress 5.1.0.41 software.

Cell proliferation was measured by the fluorescence intensity of an incorporated nuclear dye. The output is referred to as the relative cell count, where the measured nuclear intensity is transformed to percent of control (POC) using the following formula:

$$POC = \frac{{I}_{x}}{{I}_{0}} \times 100$$

Where Iₓ is the nuclear intensity at concentration x, an I₀ is the average nuclear intensity of the untreated vehicle wells.

Cellular response parameters were calculated using nonlinear regression to a sigmoidal single-site dose response model:

$$y = A + \frac{B - A}{1 + {( \frac{C}{x} )}^{D}}$$

Where y is a response measured at concentration x, A and B are the lower and upper limits of the response, C is the concentration at the response midpoint (EC₅₀), and D is the Hill Slope (Ref. 1).

Time zero non-treated plates were used to determine the number of doublings during the assay period, using the formula:

$$Doublings = {log}_{2} ( \frac{N}{{N}_{T 0}} )$$

Where N is the cell number in untreated wells at the assay end point and N_{T0} is the cell number at the time of compound addition.

Cell count IC₅₀ is the test compound concentration at 50% of maximal possible response. EC₅₀ is the test compound concentration at the curve inflection point or half the effective response (parameter C of the fitted curve solution). GI₅₀ is the concentration needed to reduce the observed growth by half (midway between the curve maximum and the time zero value). “Cell Count Activity Area” is an estimate of the integrated area above the curve (Barretina, et al. The Cancer Cell Line Encyclopedia enables predictive modelling of anticancer drug sensitivity. Nature 483: 603-607). Cell Count Activity Area values range from 0-10, where a value of zero indicates no inhibition of proliferation at all concentrations, and a value of 10 indicates complete inhibition of proliferation at all concentrations. In rare instances, values <0 or >10 may be observed. In these instances, values <0 should be considered as equivalent to 0, whereas values >10 should be considered equivalent to 10. Curve-fitting, calculations, and report generation was performed using a custom data reduction engine and MathIQ based software (AIM). Vehicle background effects on the tested cell lines are summarized in Table 1:

TABLE 1

| | | Relative cell count (POC) | | | |
|---|---|---|---|---|---|
| Plate # | Cell line | Mean | StdDev | CV | Doublings |
| 6 | SCaBER | 100.00 | 11.86 | 0.12 | 3.26 |
| 6 | SHP-77 | 100.00 | 4.45 | 0.04 | 1.68 |
| 6 | SK-N-AS | 100.00 | 4.84 | 0.05 | 1.96 |
| 6 | SNU-C2B | 100.00 | 7.16 | 0.07 | 1.40 |
| 6 | SK-NEP-1 | 100.00 | 4.59 | 0.05 | 1.49 |
| 6 | RKOE6 | 100.00 | 5.06 | 0.05 | 3.08 |
| 6 | OVCAR3 | 100.00 | 6.42 | 0.06 | 1.38 |
| 6 | NCI-H510A | 100.00 | 16.66 | 0.17 | 1.25 |
| 6 | RL95-2 | 100.00 | 3.30 | 0.03 | 1.30 |
| 6 | PC-3 | 100.00 | 6.83 | 0.07 | 2.67 |
| 6 | U-138MG | 100.00 | 5.16 | 0.05 | 1.31 |
| 6 | U2OS | 100.00 | 5.76 | 0.06 | 2.74 |
| 6 | TF-1 | 100.00 | 5.05 | 0.05 | 1.69 |
| 6 | U-118 MG | 100.00 | 7.09 | 0.07 | 1.12 |
| 6 | U-87 MG | 100.00 | 4.88 | 0.05 | 1.54 |
| 6 | ST486 | 100.00 | 6.54 | 0.07 | 1.29 |
| 6 | SK-UT-1 | 100.00 | 6.08 | 0.06 | 2.77 |
| 6 | SNU-5 | 100.00 | 1.50 | 0.01 | 1.40 |
| 6 | T24 | 100.00 | 6.03 | 0.06 | 3.44 |
| 6 | SW579 | 100.00 | 11.79 | 0.12 | 2.60 |
| 6 | A101D | 100.00 | 4.54 | 0.05 | 1.61 |
| 6 | A388 | 100.00 | 6.62 | 0.07 | 2.36 |
| 6 | Hs 852.T | 100.00 | 7.60 | 0.08 | 1.49 |
| 6 | AGS | 100.00 | 10.76 | 0.11 | 3.47 |
| 6 | A431 | 100.00 | 7.87 | 0.08 | 3.50 |
| 6 | NCIH446 | 100.00 | 10.59 | 0.11 | 1.19 |
| 6 | SW684 | 100.00 | 9.07 | 0.09 | 0.92 |
| 6 | G-402 | 100.00 | 17.97 | 0.18 | 2.46 |
| 6 | 786-O | 100.00 | 4.33 | 0.04 | 3.16 |
| 6 | Hs 729 | 100.00 | 5.22 | 0.05 | 0.85 |
| 6 | CaOV3 | 100.00 | 4.06 | 0.04 | 1.79 |
| 6 | DMS114 | 100.00 | 3.42 | 0.03 | 0.72 |
| 6 | CML-T1 | 100.00 | 2.11 | 0.02 | 2.77 |
| 6 | MES-SA | 100.00 | 3.83 | 0.04 | 3.26 |
| 6 | SK-MEL-28 | 100.00 | 3.83 | 0.04 | 1.63 |
| 6 | AsPC-1 | 100.00 | 4.21 | 0.04 | 1.20 |
| 6 | CAMA-1 | 100.00 | 6.44 | 0.06 | 1.44 |
| 6 | CRO-AP2 | 100.00 | 2.97 | 0.03 | 3.37 |
| 6 | CHP-212 | 100.00 | 2.81 | 0.03 | 1.21 |
| 6 | CEM-C1 | 100.00 | 4.09 | 0.04 | 3.06 |

The results of these assays performed using staurosporine, rapamycin, and the compound of Formula I are shown in Table 2.

TABLE 2

| | | Cell Count | Cell Count | Cell Count | Cell Count |
|---|---|---|---|---|---|
| | | EC50 | IC50 | GI50 | Activity |
| Compound | Cell Line | (microM) | (microM) | (microM) | Area |
| Staurosporine | G-402 | 9.42E−04 | 1.23E−03 | 6.64E−04 | 5.93 |
| Rapamycin | G-402 | 1.11E−04 | 3.34E−04 | 1.82E−04 | 4.68 |
| Formula I | G-402 | 1.33E−04 | 9.56E−04 | 3.32E−04 | 4.95 |
| Staurosporine | NCIH446 | 3.26E−02 | 5.02E−02 | 1.68E−02 | 2.81 |
| Rapamycin | NCIH446 | >1.00E+00 | >1.00E+00 | 4.44E−02 | 1.69 |
| Formula I | NCIH446 | 1.37E−03 | >1.00E+00 | 7.55E−02 | 1.96 |
| Staurosporine | SW684 | 1.15E−03 | >1.00E+00 | 9.50E−04 | 3.15 |
| Rapamycin | SW684 | 2.23E−03 | >1.00E+00 | 1.97E−03 | 2.81 |
| Formula I | SW684 | 4.77E−04 | 2.11E−02 | 2.86E−04 | 3.93 |
| Staurosporine | Hs 729 | 2.06E−03 | 7.04E−03 | 8.98E−04 | 4.06 |
| Rapamycin | Hs 729 | >1.00E+00 | >1.00E+00 | >1.00E+00 | 0.55 |
| Formula I | Hs 729 | 1.41E−02 | >1.00E+00 | 1.73E−02 | 1.80 |
| Staurosporine | 786-O | 3.58E−03 | 4.33E−03 | 3.41E−03 | 4.87 |
| Rapamycin | 786-O | 2.27E−04 | 3.18E−03 | 1.13E−03 | 4.17 |
| Formula I | 786-O | 3.95E−04 | 4.07E−03 | 1.91E−03 | 4.43 |
| Staurosporine | Hs 852.T | 9.30E−04 | 5.85E−03 | 1.35E−03 | 3.16 |
| Rapamycin | Hs 852.T | >1.00E+00 | >1.00E+00 | >1.00E+00 | 1.43 |
| Formula I | Hs 852.T | 3.35E−01 | >1.00E+00 | 8.03E−01 | 1.22 |
| Staurosporine | A101D | 7.02E−03 | 8.74E−03 | 2.65E−03 | 4.56 |
| Rapamycin | A101D | 1.84E−04 | >1.00E+00 | 7.21E−04 | 2.92 |
| Formula I | A101D | 3.22E−04 | >1.00E+00 | 9.99E−04 | 3.52 |
| Staurosporine | A388 | 9.17E−04 | 1.62E−03 | 1.03E−03 | 4.99 |
| Rapamycin | A388 | >1.00E+00 | >1.00E+00 | >1.00E+00 | 2.25 |
| Formula I | A388 | 1.90E−04 | >1.00E+00 | 5.07E−02 | 3.22 |
| Staurosporine | A431 | 9.53E−04 | 1.19E−03 | 1.03E−03 | 5.64 |
| Rapamycin | A431 | >1.00E+00 | >1.00E+00 | >1.00E+00 | 0.28 |
| Formula I | A431 | 1.43E−02 | >1.00E+00 | >1.00E+00 | 1.96 |
| Staurosporine | AGS | 1.16E−03 | 1.21E−03 | 9.26E−04 | 6.19 |
| Rapamycin | AGS | 3.87E−05 | 3.14E−04 | 1.65E−04 | 5.67 |
| Formula I | AGS | 6.35E−05 | 1.86E−04 | 1.36E−04 | 5.50 |
| Staurosporine | CRO-AP2 | 1.36E−02 | 1.43E−02 | 1.32E−02 | 3.99 |
| Rapamycin | CRO-AP2 | 1.19E−04 | 1.87E−04 | 1.55E−04 | 6.22 |
| Formula I | CRO-AP2 | 3.19E−04 | 5.36E−04 | 4.34E−04 | 5.61 |
| Staurosporine | AsPC-1 | 1.99E−03 | 3.93E−03 | 2.05E−03 | 3.28 |
| Rapamycin | AsPC-1 | >1.00E+00 | >1.00E+00 | >1.00E+00 | 1.48 |
| Formula I | AsPC-1 | 5.19E−04 | >1.00E+00 | 8.07E−03 | 2.50 |
| Staurosporine | CAMA-1 | 2.02E−02 | 9.90E−02 | 1.20E−02 | 2.99 |
| Rapamycin | CAMA-1 | 1.94E−04 | 1.19E−03 | 2.25E−04 | 4.07 |
| Formula I | CAMA-1 | 1.74E−04 | 1.28E−03 | 2.39E−04 | 4.42 |
| Staurosporine | CEM-C1 | 7.60E−03 | 9.20E−03 | 7.27E−03 | 4.28 |
| Rapamycin | CEM-C1 | 2.65E−04 | >1.00E+00 | 4.36E−03 | 3.61 |
| Formula I | CEM-C1 | 5.00E−04 | 3.91E−01 | 1.02E−02 | 3.52 |
| Staurosporine | CHP-212 | 4.19E−03 | 4.78E−03 | 2.59E−03 | 4.80 |
| Rapamycin | CHP-212 | 3.76E−05 | >1.00E+00 | 7.07E−05 | 4.13 |
| Formula I | CHP-212 | 9.18E−05 | 1.93E−02 | 1.25E−04 | 3.70 |
| Staurosporine | CML-T1 | 7.06E−03 | 1.02E−02 | 6.96E−03 | 4.13 |
| Rapamycin | CML-T1 | 1.89E−04 | 1.93E−03 | 6.94E−04 | 4.56 |
| Formula I | CML-T1 | 3.17E−04 | 2.51E−03 | 9.98E−04 | 4.54 |
| Staurosporine | CaOV3 | 2.44E−04 | 5.39E−04 | 2.73E−04 | 5.31 |
| Rapamycin | CaOV3 | >1.00E+00 | >1.00E+00 | >1.00E+00 | 2.40 |
| Formula I | CaOV3 | 9.76E−04 | >1.00E+00 | 9.68E−03 | 2.82 |
| Staurosporine | DMS114 | 2.10E−02 | 5.68E−02 | 3.35E−03 | 3.04 |
| Rapamycin | DMS114 | 7.47E−05 | >1.00E+00 | 4.78E−05 | 4.14 |
| Formula I | DMS114 | 2.51E−04 | 4.40E−03 | 8.22E−05 | 4.35 |
| Staurosporine | SK-MEL-28 | 5.39E−02 | 6.43E−02 | 3.21E−02 | 2.81 |
| Rapamycin | SK-MEL-28 | 6.54E−04 | >1.00E+00 | 9.87E−02 | 2.50 |
| Formula I | SK-MEL-28 | 1.94E−04 | >1.00E+00 | >1.00E+00 | 1.87 |
| Staurosporine | MES-SA | 1.25E−02 | 1.42E−02 | 1.30E−02 | 2.80 |
| Rapamycin | MES-SA | 1.74E−04 | 4.15E−04 | 2.94E−04 | 5.39 |
| Formula I | MES-SA | 3.22E−04 | 6.44E−04 | 4.95E−04 | 5.42 |
| Staurosporine | NCI-H510A | 3.98E−02 | >1.00E+00 | 5.65E−02 | 1.68 |
| Rapamycin | NCI-H510A | >1.00E+00 | >1.00E+00 | >1.00E+00 | 1.63 |
| Formula I | NCI-H510A | 3.99E−06 | >1.00E+00 | 8.30E−04 | 3.05 |
| Staurosporine | RKOE6 | 1.68E−02 | 1.79E−02 | 1.32E−02 | 3.96 |
| Rapamycin | RKOE6 | 8.35E−05 | 1.06E−03 | 3.56E−04 | 4.61 |
| Formula I | RKOE6 | 2.01E−04 | 2.88E−03 | 1.07E−03 | 4.44 |
| Staurosporine | OVCAR3 | 7.87E−03 | 1.73E−02 | 6.11E−03 | 3.41 |
| Rapamycin | OVCAR3 | 1.53E−03 | >1.00E+00 | 1.24E−01 | 2.08 |
| Formula I | OVCAR3 | 6.46E−01 | >1.00E+00 | 6.58E−02 | 2.43 |
| Staurosporine | PC-3 | 9.03E−04 | 1.31E−03 | 1.07E−03 | 4.67 |
| Rapamycin | PC-3 | 2.30E−04 | >1.00E+00 | 2.63E−03 | 3.57 |
| Formula I | PC-3 | 3.46E−04 | 9.55E−03 | 1.89E−03 | 3.97 |
| Staurosporine | RL95-2 | 1.86E−02 | 2.01E−02 | 6.83E−03 | 3.87 |
| Rapamycin | RL95-2 | 7.05E−05 | >1.00E+00 | 1.30E−04 | 4.24 |
| Formula I | RL95-2 | 1.50E−04 | 1.09E−02 | 2.20E−04 | 4.12 |
| Staurosporine | SK-N-AS | 1.70E−03 | 3.86E−03 | 1.27E−03 | 4.83 |
| Rapamycin | SK-N-AS | 2.31E−04 | >1.00E+00 | 4.32E−04 | 3.58 |
| Formula I | SK-N-AS | 2.83E−04 | 7.35E−03 | 8.26E−04 | 3.99 |
| Staurosporine | SCaBER | 4.22E−04 | 5.45E−04 | 4.74E−04 | 6.03 |
| Rapamycin | SCaBER | 3.77E−05 | 3.21E−04 | 1.58E−04 | 5.25 |
| Formula I | SCaBER | 9.47E−05 | 4.51E−04 | 2.62E−04 | 5.46 |
| Staurosporine | SHP-77 | 2.81E−02 | 3.08E−02 | 2.42E−02 | 3.16 |
| Rapamycin | SHP-77 | >1.00E+00 | >1.00E+00 | >1.00E+00 | 1.13 |
| Formula I | SHP-77 | 3.09E−01 | >1.00E+00 | >1.00E+00 | 1.18 |
| Staurosporine | SK-NEP-1 | 4.82E−02 | 5.45E−02 | 3.65E−02 | 2.78 |
| Rapamycin | SK-NEP-1 | 2.93E−04 | 1.47E−03 | 3.61E−04 | 4.22 |
| Formula I | SK-NEP-1 | 7.06E−04 | 2.42E−03 | 7.47E−04 | 4.35 |
| Staurosporine | SNU-C2B | 1.58E−02 | 2.47E−02 | 4.97E−03 | 3.80 |
| Rapamycin | SNU-C2B | >1.00E+00 | >1.00E+00 | >1.00E+00 | 1.99 |
| Formula I | SNU-C2B | 2.07E−03 | >1.00E+00 | 2.41E−02 | 2.30 |
| Staurosporine | SNU-5 | 6.44E−03 | 2.28E−01 | 1.03E−02 | 2.58 |
| Rapamycin | SNU-5 | 8.85E−04 | >1.00E+00 | 2.40E−02 | 2.23 |
| Formula I | SNU-5 | 2.05E−03 | >1.00E+00 | 2.38E−02 | 2.14 |
| Staurosporine | ST486 | 4.22E−03 | 4.61E−03 | 2.83E−03 | 4.89 |
| Rapamycin | ST486 | 3.54E−05 | >1.00E+00 | 5.24E−05 | 3.99 |
| Formula I | ST486 | 1.28E−04 | >1.00E+00 | 2.26E−04 | 3.70 |
| Staurosporine | SK-UT-1 | 2.18E−02 | 2.86E−02 | 1.95E−02 | 3.46 |
| Rapamycin | SK-UT-1 | 2.00E−04 | >1.00E+00 | >1.00E+00 | 3.23 |
| Formula I | SK-UT-1 | 2.84E−04 | 1.17E−02 | 2.01E−03 | 3.95 |
| Staurosporine | SW579 | 5.20E−04 | 6.69E−04 | 5.06E−04 | 6.09 |
| Rapamycin | SW579 | 1.36E−05 | >1.00E+00 | 5.39E−05 | 4.77 |
| Formula I | SW579 | 6.16E−05 | 3.67E−04 | 1.52E−04 | 5.31 |
| Staurosporine | T24 | 8.01E−04 | 9.53E−04 | 8.29E−04 | 5.96 |
| Rapamycin | T24 | 7.83E−05 | 2.10E−03 | 4.74E−04 | 4.39 |
| Formula I | T24 | 1.02E−04 | 9.05E−04 | 4.75E−04 | 4.92 |
| Staurosporine | TF-1 | 1.08E−02 | 1.19E−02 | 1.09E−02 | 3.31 |
| Rapamycin | TF-1 | >1.00E+00 | >1.00E+00 | >1.00E+00 | 1.52 |
| Formula I | TF-1 | >1.00E+00 | >1.00E+00 | >1.00E+00 | 1.64 |
| Staurosporine | U-138MG | 8.57E−04 | 5.44E−03 | 1.05E−03 | 3.72 |
| Rapamycin | U-138MG | 3.63E−05 | >1.00E+00 | 4.36E−04 | 3.14 |
| Formula I | U-138MG | 9.75E−05 | >1.00E+00 | 5.40E−04 | 3.00 |
| Staurosporine | U2OS | 2.72E−03 | 3.40E−03 | 2.63E−03 | 4.91 |
| Rapamycin | U2OS | 6.50E−04 | >1.00E+00 | 2.54E−03 | 3.04 |
| Formula I | U2OS | 3.95E−04 | 1.21E−02 | 2.76E−03 | 4.05 |
| Staurosporine | U-87 MG | 7.52E−04 | 2.46E−03 | 8.36E−04 | 3.25 |
| Rapamycin | U-87 MG | 1.04E−04 | >1.00E+00 | 3.84E−04 | 3.79 |
| Formula I | U-87 MG | 2.06E−04 | 4.88E−02 | 5.09E−04 | 3.92 |
| Staurosporine | U-118 MG | 1.00E−03 | >1.00E+00 | 1.93E−03 | 3.04 |
| Rapamycin | U-118 MG | 7.92E−05 | >1.00E+00 | 1.24E−04 | 2.87 |
| Formula I | U-118 MG | 1.89E−04 | >1.00E+00 | 5.57E−04 | 3.03 |

A further detailed analysis of the effect of the compound of Formula I on proliferation of each cell line is provided in the Table 3 below:

TABLE 3

Relative cell count (%)

| | | |
|---|---|---|
| Concentration (microM) | Mean | StdDev |

G-402
Human renal leiomyoblastoma

| | | |
|---|---|---|
| 3.18E−05 | 89.9 | 3.9 |
| 1.01E−04 | 70.9 | 5.1 |
| 3.18E−04 | 54.9 | 4.5 |
| 1.00E−03 | 49.2 | 6.2 |
| 3.17E−03 | 48.8 | 1.2 |
| 1.00E−02 | 41.4 | 2.0 |
| 3.17E−02 | 46.6 | 6.4 |
| 1.00E−01 | 37.2 | 10.5 |
| 3.16E−01 | 34.9 | 11.1 |
| 1.00E+00 | 31.5 | 8.9 |

NCIH446
Small cell lung carcinoma

| | | |
|---|---|---|
| 3.18E−05 | 99.4 | 3.9 |
| 1.01E−04 | 102.8 | 11.1 |
| 3.18E−04 | 100.1 | 9.9 |
| 1.00E−03 | 88.1 | 6.0 |
| 3.17E−03 | 82.3 | 12.7 |
| 1.00E−02 | 75.9 | 1.4 |
| 3.17E−02 | 85.0 | 5.8 |
| 1.00E−01 | 77.0 | 7.7 |
| 3.16E−01 | 71.2 | 18.1 |
| 1.00E+00 | 69.7 | 5.2 |

SW684
Human fibrosarcoma

| | | |
|---|---|---|
| 3.18E−05 | 91.9 | 3.2 |
| 1.01E−04 | 89.3 | 2.7 |
| 3.18E−04 | 77.8 | 5.9 |
| 1.00E−03 | 68.8 | 3.4 |
| 3.17E−03 | 60.7 | 3.4 |
| 1.00E−02 | 55.0 | 3.4 |
| 3.17E−02 | 52.7 | 3.2 |
| 1.00E−01 | 51.0 | 1.2 |
| 3.16E−01 | 47.1 | 2.3 |
| 1.00E+00 | 44.1 | 1.4 |

Hs 729
Human rhabdomyosarcoma

| | | |
|---|---|---|
| 3.18E−05 | 91.5 | 2.9 |
| 1.01E−04 | 95.7 | 0.4 |
| 3.18E−04 | 87.7 | 1.0 |
| 1.00E−03 | 87.3 | 1.5 |
| 3.17E−03 | 79.1 | 3.2 |
| 1.00E−02 | 79.6 | 1.0 |
| 3.17E−02 | 76.9 | 1.8 |
| 1.00E−01 | 69.6 | 1.8 |
| 3.16E−01 | 63.1 | 8.9 |
| 1.00E+00 | 65.5 | 2.7 |

786-0
Human renal cell adenocarcinoma

| | | |
|---|---|---|
| 3.18E−05 | 100.6 | 8.6 |
| 1.01E−04 | 88.5 | 2.7 |
| 3.18E−04 | 77.4 | 3.3 |
| 1.00E−03 | 61.1 | 2.6 |
| 3.17E−03 | 60.0 | 0.6 |
| 1.00E−02 | 52.1 | 2.6 |
| 3.17E−02 | 46.5 | 3.3 |
| 1.00E−01 | 44.8 | 3.0 |
| 3.16E−01 | 43.9 | 3.1 |
| 1.00E+00 | 37.4 | 3.1 |

Hs 852.T
Human melanoma

| | | |
|---|---|---|
| 3.18E−05 | 99.0 | 4.1 |
| 1.01E−04 | 106.1 | 2.2 |
| 3.18E−04 | 105.1 | 3.2 |
| 1.00E−03 | 96.5 | 7.4 |
| 3.17E−03 | 100.5 | 1.6 |
| 1.00E−02 | 92.9 | 6.4 |
| 3.17E−02 | 87.7 | 1.4 |
| 1.00E−01 | 82.5 | 3.0 |
| 3.16E−01 | 78.0 | 3.9 |
| 1.00E+00 | 67.7 | 6.5 |

A101D
Human melanoma

| | | |
|---|---|---|
| 3.18E−05 | 95.8 | 7.0 |
| 1.01E−04 | 90.7 | 10.0 |
| 3.18E−04 | 78.1 | 4.5 |
| 1.00E−03 | 66.8 | 6.5 |
| 3.17E−03 | 61.9 | 4.7 |
| 1.00E−02 | 54.7 | 2.6 |
| 3.17E−02 | 58.0 | 2.9 |
| 1.00E−01 | 57.8 | 2.1 |
| 3.16E−01 | 52.8 | 3.2 |
| 1.00E+00 | 50.8 | 3.0 |

A388
Human epidermoid carcinoma

| | | |
|---|---|---|
| 3.18E−05 | 89.9 | 0.1 |
| 1.01E−04 | 77.9 | 2.3 |
| 3.18E−04 | 76.3 | 5.1 |
| 1.00E−03 | 69.1 | 5.7 |
| 3.17E−03 | 67.1 | 1.2 |
| 1.00E−02 | 63.3 | 1.6 |
| 3.17E−02 | 59.6 | 3.9 |
| 1.00E−01 | 62.3 | 7.5 |
| 3.16E−01 | 59.0 | 2.4 |
| 1.00E+00 | 53.8 | 6.2 |

A431
Human epidermoid carcinoma

| | | |
|---|---|---|
| 3.18E−05 | 104.6 | 15.2 |
| 1.01E−04 | 101.0 | 5.8 |
| 3.18E−04 | 91.9 | 10.0 |
| 1.00E−03 | 93.5 | 3.5 |
| 3.17E−03 | 84.4 | 2.7 |
| 1.00E−02 | 84.2 | 14.5 |
| 3.17E−02 | 78.2 | 4.9 |
| 1.00E−01 | 72.8 | 4.0 |
| 3.16E−01 | 66.5 | 5.5 |
| 1.00E+00 | 64.2 | 6.7 |

AGS
Human gastric adenocarcinoma

| | | |
|---|---|---|
| 3.18E−05 | 82.7 | 4.9 |
| 1.01E−04 | 58.2 | 5.8 |
| 3.18E−04 | 45.6 | 9.1 |
| 1.00E−03 | 39.8 | 0.3 |
| 3.17E−03 | 40.1 | 1.3 |
| 1.00E−02 | 39.1 | 4.1 |
| 3.17E−02 | 34.7 | 2.8 |
| 1.00E−01 | 39.3 | 7.6 |
| 3.16E−01 | 38.3 | 1.5 |
| 1.00E+00 | 32.3 | 3.9 |

CRO-AP2
Human B cell lymphoma

| | | |
|---|---|---|
| 3.18E−05 | 93.4 | 4.1 |
| 1.01E−04 | 89.0 | 3.3 |
| 3.18E−04 | 59.0 | 5.5 |
| 1.00E−03 | 38.7 | 0.8 |
| 3.17E−03 | 29.7 | 1.6 |
| 1.00E−02 | 27.9 | 0.4 |
| 3.17E−02 | 23.6 | 0.4 |
| 1.00E−01 | 24.5 | 1.6 |
| 3.16E−01 | 23.3 | 2.2 |
| 1.00E+00 | 23.3 | 1.6 |

AsPC-1
Human adenocarcinoma

| | | |
|---|---|---|
| 3.18E−05 | 99.5 | 4.9 |
| 1.01E−04 | 89.6 | 2.8 |
| 3.18E−04 | 94.5 | 12.5 |
| 1.00E−03 | 81.7 | 8.1 |
| 3.17E−03 | 77.8 | 3.8 |
| 1.00E−02 | 68.9 | 2.1 |
| 3.17E−02 | 74.7 | 2.9 |
| 1.00E−01 | 74.0 | 5.5 |
| 3.16E−01 | 67.9 | 3.5 |
| 1.00E+00 | 64.8 | 5.0 |

CAMA-1
Human breast adenocarcinoma

| | | |
|---|---|---|
| 3.18E−05 | 97.5 | 5.4 |
| 1.01E−04 | 86.0 | 3.9 |
| 3.18E−04 | 66.1 | 2.7 |
| 1.00E−03 | 53.9 | 3.4 |
| 3.17E−03 | 50.2 | 1.3 |
| 1.00E−02 | 51.2 | 1.6 |
| 3.17E−02 | 48.8 | 2.9 |
| 1.00E−01 | 45.8 | 1.5 |
| 3.16E−01 | 46.2 | 3.8 |
| 1.00E+00 | 44.1 | 3.2 |

CEM-C1
Human acute lymphoblastic leukemia

| | | |
|---|---|---|
| 3.18E−05 | 94.0 | 0.7 |
| 1.01E−04 | 90.0 | 5.3 |
| 3.18E−04 | 72.7 | 7.1 |
| 1.00E−03 | 68.7 | 3.2 |
| 3.17E−03 | 62.4 | 2.2 |
| 1.00E−02 | 58.4 | 1.8 |
| 3.17E−02 | 53.1 | 1.0 |
| 1.00E−01 | 53.2 | 2.7 |
| 3.16E−01 | 48.9 | 1.7 |
| 1.00E+00 | 47.1 | 1.8 |

CHP-212
Human neuroblastoma

| | | |
|---|---|---|
| 3.18E−05 | 86.7 | 3.7 |
| 1.01E−04 | 70.6 | 7.7 |
| 3.18E−04 | 63.3 | 3.9 |
| 1.00E−03 | 56.6 | 1.4 |
| 3.17E−03 | 55.3 | 2.1 |
| 1.00E−02 | 52.8 | 3.3 |
| 3.17E−02 | 49.0 | 3.1 |
| 1.00E−01 | 49.6 | 1.5 |
| 3.16E−01 | 45.9 | 3.1 |
| 1.00E+00 | N/A | N/A |

CML-T1
Human chronic myelogenous leukemia

| | | |
|---|---|---|
| 3.18E−05 | 90.6 | 3.5 |
| 1.01E−04 | 83.6 | 3.4 |
| 3.18E−04 | 65.3 | 3.7 |
| 1.00E−03 | 55.6 | 3.0 |
| 3.17E−03 | 49.5 | 2.5 |
| 1.00E−02 | 46.7 | 1.8 |
| 3.17E−02 | 42.3 | 3.6 |
| 1.00E−01 | 42.6 | 3.6 |
| 3.16E−01 | 33.1 | 2.9 |
| 1.00E+00 | 36.5 | 2.2 |

CaOV3
Human ovary adenocarcinoma

| | | |
|---|---|---|
| 3.18E−05 | 92.8 | 6.1 |
| 1.01E−04 | 91.0 | 3.4 |
| 3.18E−04 | 85.0 | 7.1 |
| 1.00E−03 | 75.3 | 1.6 |
| 3.17E−03 | 68.5 | 6.0 |
| 1.00E−02 | 64.8 | 7.3 |
| 3.17E−02 | 59.5 | 2.3 |
| 1.00E−01 | 58.3 | 4.7 |
| 3.16E−01 | 57.1 | 1.5 |
| 1.00E+00 | 54.4 | 2.3 |

DMS114
Human lung carcinoma

| | | |
|---|---|---|
| 3.18E−05 | 90.1 | 5.0 |
| 1.01E−04 | 77.8 | 5.8 |
| 3.18E−04 | 67.2 | 0.6 |
| 1.00E−03 | 57.7 | 5.5 |
| 3.17E−03 | 51.7 | 1.9 |
| 1.00E−02 | 50.7 | 1.5 |
| 3.17E−02 | 44.7 | 1.4 |
| 1.00E−01 | 44.1 | 0.7 |
| 3.16E−01 | 41.4 | 2.4 |
| 1.00E+00 | 39.3 | 0.3 |

SK-MEL-28
Human malignant melanoma

| | | |
|---|---|---|
| 3.18E−05 | 93.8 | 6.8 |
| 1.01E−04 | 88.2 | 5.4 |
| 3.18E−04 | 79.6 | 6.4 |
| 1.00E−03 | 75.0 | 2.3 |
| 3.17E−03 | 70.8 | 3.4 |
| 1.00E−02 | 71.4 | 0.3 |
| 3.17E−02 | 68.9 | 0.5 |
| 1.00E−01 | 65.6 | 4.7 |
| 3.16E−01 | N/A | N/A |
| 1.00E+00 | N/A | N/A |

MES-SA
Human uterine sarcoma

| | | |
|---|---|---|
| 3.18E−05 | 98.1 | 2.7 |
| 1.01E−04 | 93.8 | 4.6 |
| 3.18E−04 | 64.3 | 10.0 |
| 1.00E−03 | 45.0 | 5.7 |
| 3.17E−03 | 37.3 | 2.8 |
| 1.00E−02 | 32.0 | 4.3 |
| 3.17E−02 | 28.6 | 2.2 |
| 1.00E−01 | 28.9 | 3.5 |
| 3.16E−01 | 26.8 | 1.0 |
| 1.00E+00 | 27.9 | 1.5 |

NCI-H510A
Human metastatic small cell lung cancer

| | | |
|---|---|---|
| 3.18E−05 | 75.7 | 2.9 |
| 1.01E−04 | 77.7 | 7.6 |
| 3.18E−04 | 70.5 | 5.5 |
| 1.00E−03 | 68.9 | 6.2 |
| 3.17E−03 | 72.2 | 5.0 |
| 1.00E−02 | 69.5 | 2.7 |
| 3.17E−02 | 64.7 | 1.5 |
| 1.00E−01 | 62.8 | 5.5 |
| 3.16E−01 | 66.3 | 0.5 |
| 1.00E+00 | 66.2 | 1.6 |

RKOE6
Human epithelial carcinoma (colon)

| | | |
|---|---|---|
| 3.18E−05 | 92.5 | 6.3 |
| 1.01E−04 | 73.6 | 6.0 |
| 3.18E−04 | 66.1 | 5.6 |
| 1.00E−03 | 55.7 | 4.6 |
| 3.17E−03 | 52.5 | 0.9 |
| 1.00E−02 | 47.6 | 6.1 |
| 3.17E−02 | 42.9 | 2.3 |
| 1.00E−01 | 43.0 | 3.8 |
| 3.16E−01 | 41.0 | 4.6 |
| 1.00E+00 | 41.6 | 4.3 |

OVCAR3
Human ovary adenocarcinoma

| | | |
|---|---|---|
| 3.18E−05 | 106.9 | 2.3 |
| 1.01E−04 | 93.3 | 4.0 |
| 3.18E−04 | 89.7 | 3.1 |
| 1.00E−03 | 90.2 | 4.5 |
| 3.17E−03 | 85.2 | 4.5 |
| 1.00E−02 | 85.0 | 4.2 |
| 3.17E−02 | 79.9 | 0.9 |
| 1.00E−01 | 72.8 | 3.9 |
| 3.16E−01 | 61.7 | 2.4 |
| 1.00E+00 | 61.8 | 4.8 |

PC-3
Human prostate adenocarcinoma

| | | |
|---|---|---|
| 3.18E−05 | 93.7 | 5.0 |
| 1.01E−04 | 87.3 | 4.7 |
| 3.18E−04 | 69.1 | 6.4 |
| 1.00E−03 | 59.2 | 2.1 |
| 3.17E−03 | 59.0 | 3.0 |
| 1.00E−02 | 53.6 | 5.8 |
| 3.17E−02 | 47.1 | 1.6 |
| 1.00E−01 | 47.9 | 4.8 |
| 3.16E−01 | 43.8 | 1.9 |
| 1.00E+00 | 42.2 | 1.8 |

RL95-2
Human uterus carcinoma

| | | |
|---|---|---|
| 3.18E−05 | 89.3 | 3.8 |
| 1.01E−04 | 78.3 | 3.4 |
| 3.18E−04 | 64.0 | 5.9 |
| 1.00E−03 | 59.5 | 3.6 |
| 3.17E−03 | 52.9 | 1.7 |
| 1.00E−02 | 52.2 | 2.5 |
| 3.17E−02 | 48.5 | 2.9 |
| 1.00E−01 | 50.6 | 3.0 |
| 3.16E−01 | 45.6 | 3.9 |
| 1.00E+00 | 47.2 | 3.2 |

SK-N-AS
Human neuroblastoma

| | | |
|---|---|---|
| 3.18E−05 | 102.1 | 3.2 |
| 1.01E−04 | 84.8 | 6.6 |
| 3.18E−04 | 77.7 | 9.9 |
| 1.00E−03 | 62.3 | 5.5 |
| 3.17E−03 | 56.7 | 8.9 |
| 1.00E−02 | 53.6 | 1.0 |
| 3.17E−02 | 52.4 | 4.0 |
| 1.00E−01 | 51.0 | 4.7 |
| 3.16E−01 | 48.3 | 1.5 |
| 1.00E+00 | 45.8 | 2.7 |

SCaBER
Human squamous cell carcinoma (bladder)

| | | |
|---|---|---|
| 3.18E−05 | 81.9 | 6.5 |
| 1.01E−04 | 61.7 | 7.1 |
| 3.18E−04 | 55.4 | 8.4 |
| 1.00E−03 | 44.7 | 2.3 |
| 3.17E−03 | 38.7 | 3.9 |
| 1.00E−02 | 35.4 | 4.0 |
| 3.17E−02 | 34.0 | 3.4 |
| 1.00E−01 | 36.9 | 3.6 |
| 3.16E−01 | 33.0 | 1.6 |
| 1.00E+00 | 32.4 | 3.0 |

SHP-77
Human small cell lung carcinoma

| | | |
|---|---|---|
| 3.18E−05 | 99.1 | 5.0 |
| 1.01E−04 | 98.0 | 1.2 |
| 3.18E−04 | 101.7 | 6.5 |
| 1.00E−03 | 96.5 | 4.1 |
| 3.17E−03 | 89.5 | 1.3 |
| 1.00E−02 | 94.0 | 6.9 |
| 3.17E−02 | 84.9 | 3.2 |
| 1.00E−01 | 83.4 | 1.3 |
| 3.16E−01 | 75.9 | 3.7 |
| 1.00E+00 | 71.6 | 5.1 |

SK-NEP-1
Human metastatic kidney cancer

| | | |
|---|---|---|
| 3.18E−05 | 99.4 | 1.5 |
| 1.01E−04 | 100.0 | 2.3 |
| 3.18E−04 | 83.9 | 3.2 |
| 1.00E−03 | 63.5 | 3.0 |
| 3.17E−03 | 48.8 | 1.1 |
| 1.00E−02 | 45.3 | 2.4 |
| 3.17E−02 | 40.0 | 2.8 |
| 1.00E−01 | 38.6 | 2.8 |
| 3.16E−01 | 35.6 | 0.7 |
| 1.00E+00 | 35.6 | 4.1 |

SNU-C2B
Human colorectal carcinoma

| | | |
|---|---|---|
| 3.18E−05 | 83.2 | 2.6 |
| 1.01E−04 | 89.1 | 7.7 |
| 3.18E−04 | 79.6 | 0.9 |
| 1.00E−03 | 73.7 | 17.1 |
| 3.17E−03 | 71.6 | 5.1 |
| 1.00E−02 | 68.6 | 8.2 |
| 3.17E−02 | 63.2 | 8.1 |
| 1.00E−01 | 65.6 | 2.9 |
| 3.16E−01 | 55.1 | 3.2 |
| 1.00E+00 | 58.7 | 10.8 |

SNU-5
Human metastatic gastric carcinoma

| | | |
|---|---|---|
| 3.18E−05 | 96.3 | 1.0 |
| 1.01E−04 | 95.3 | 1.5 |
| 3.18E−04 | 91.2 | 2.3 |
| 1.00E−03 | 84.5 | 0.9 |
| 3.17E−03 | 77.6 | 0.5 |
| 1.00E−02 | 73.2 | 1.1 |
| 3.17E−02 | 65.8 | 1.7 |
| 1.00E−01 | 65.7 | 1.2 |
| 3.16E−01 | 64.0 | 1.3 |
| 1.00E+00 | 62.8 | 1.7 |

ST486
Human Burkitt's Lymphoma (B cell)

| | | |
|---|---|---|
| 3.18E−05 | 90.7 | 1.8 |
| 1.01E−04 | 81.6 | 8.6 |
| 3.18E−04 | 63.3 | 8.1 |
| 1.00E−03 | 61.1 | 8.4 |
| 3.17E−03 | 56.0 | 3.8 |
| 1.00E−02 | 56.8 | 1.2 |
| 3.17E−02 | 54.8 | 5.8 |
| 1.00E−01 | 58.5 | 4.1 |
| 3.16E−01 | 51.1 | 3.0 |
| 1.00E+00 | 50.5 | 4.1 |

SK-UT-1
Human uterine tumor

| | | |
|---|---|---|
| 3.18E−05 | 98.4 | 21.7 |
| 1.01E−04 | 94.8 | 16.0 |
| 3.18E−04 | 72.6 | 7.3 |
| 1.00E−03 | 72.8 | 4.9 |
| 3.17E−03 | 57.1 | 3.0 |
| 1.00E−02 | 52.2 | 3.2 |
| 3.17E−02 | 53.1 | 9.0 |
| 1.00E−01 | 57.6 | 2.0 |
| 3.16E−01 | 48.7 | 4.3 |
| 1.00E+00 | 50.0 | 4.2 |

SW579
Human squamous cell carcinoma

| | | |
|---|---|---|
| 3.18E−05 | 78.0 | 10.1 |
| 1.01E−04 | 63.2 | 9.2 |
| 3.18E−04 | 48.4 | 1.8 |
| 1.00E−03 | 44.2 | 2.1 |
| 3.17E−03 | 44.6 | 1.8 |
| 1.00E−02 | 42.8 | 4.2 |
| 3.17E−02 | 38.4 | 3.7 |
| 1.00E−01 | 35.8 | 0.9 |
| 3.16E−01 | 37.9 | 0.0 |
| 1.00E+00 | 35.3 | 4.4 |

T24
Human bladder transitional cell carcinoma

| | | |
|---|---|---|
| 3.18E−05 | 84.1 | 12.5 |
| 1.01E−04 | 68.7 | 4.9 |
| 3.18E−04 | 57.0 | 2.7 |
| 1.00E−03 | 49.5 | 2.8 |
| 3.17E−03 | 46.0 | 2.6 |
| 1.00E−02 | 43.9 | 2.5 |
| 3.17E−02 | 42.0 | 1.7 |
| 1.00E−01 | 41.3 | 3.1 |
| 3.16E−01 | 37.1 | 2.9 |
| 1.00E+00 | 38.7 | 3.1 |

TF-1
Human erythroleukemia (bone marrow)

| | | |
|---|---|---|
| 3.18E−05 | 96.7 | 1.8 |
| 1.01E−04 | 97.8 | 1.7 |
| 3.18E−04 | 88.0 | 1.9 |
| 1.00E−03 | 87.4 | 4.6 |
| 3.17E−03 | 88.0 | 4.9 |
| 1.00E−02 | 81.1 | 2.9 |
| 3.17E−02 | 81.3 | 6.8 |
| 1.00E−01 | 80.1 | 2.1 |
| 3.16E−01 | 80.1 | 3.7 |
| 1.00E+00 | 80.6 | 2.5 |

U-138MG
Human glioblastoma

| | | |
|---|---|---|
| 3.18E−05 | 98.2 | 5.3 |
| 1.01E−04 | 82.1 | 4.9 |
| 3.18E−04 | 75.7 | 4.4 |
| 1.00E−03 | 70.6 | 3.8 |
| 3.17E−03 | 70.9 | 0.5 |
| 1.00E−02 | 71.3 | 5.3 |
| 3.17E−02 | 66.8 | 4.9 |
| 1.00E−01 | 66.3 | 5.7 |
| 3.16E−01 | 64.5 | 1.1 |
| 1.00E+00 | 62.8 | 4.6 |

U2OS
Human osteosarcoma

| | | |
|---|---|---|
| 3.18E−05 | 95.3 | 3.5 |
| 1.01E−04 | 86.1 | 4.7 |
| 3.18E−04 | 75.4 | 10.8 |
| 1.00E−03 | 69.9 | 4.0 |
| 3.17E−03 | 62.6 | 6.2 |
| 1.00E−02 | 51.4 | 10.6 |
| 3.17E−02 | 47.8 | 4.6 |
| 1.00E−01 | 48.5 | 4.7 |
| 3.16E−01 | 43.2 | 1.8 |
| 1.00E+00 | 50.8 | 2.0 |

U-87 MG
Human brain cancer (glioblastoma)

| | | |
|---|---|---|
| 3.18E−05 | 93.7 | 0.8 |
| 1.01E−04 | 87.3 | 2.9 |
| 3.18E−04 | 71.8 | 6.4 |
| 1.00E−03 | 64.8 | 3.4 |
| 3.17E−03 | 58.9 | 5.3 |
| 1.00E−02 | 54.7 | 4.8 |
| 3.17E−02 | 50.4 | 2.9 |
| 1.00E−01 | 54.5 | 3.2 |
| 3.16E−01 | 51.6 | 0.5 |
| 1.00E+00 | 51.2 | 1.7 |

U-118 MG
Human glioblastoma

| | | |
|---|---|---|
| 3.18E−05 | 94.0 | 0.6 |
| 1.01E−04 | 84.6 | 4.5 |
| 3.18E−04 | 74.5 | 1.3 |
| 1.00E−03 | 71.7 | 5.0 |
| 3.17E−03 | 63.9 | 5.5 |
| 1.00E−02 | 63.6 | 6.6 |
| 3.17E−02 | 65.5 | 6.9 |
| 1.00E−01 | 59.7 | 5.8 |
| 3.16E−01 | 64.8 | 4.7 |
| 1.00E+00 | 55.1 | 2.5 |

A summary of this data regarding cell lines for which xenograft models are available is provided in Table 4:

TABLE 4

| Cell Line | Tissue | Type | EC₅₀ | Comment |
|---|---|---|---|---|
| A431 | Skin | Head/Neck | 1.4 × 10⁻² | Negative |
| | | | | control |
| OVACAR3 | Female GU | Ovarian | 6.5 × 10⁻¹ | mTOR over- |
| | | | | expressing |
| NCI-H510A | Lung | Small cell | 4.0 × 10⁻⁴ | mTOR over- |
| | | lung cancer | | expressing |
| | | (SCLC) | | |
| SK-MEL-82 | Skin | Melanoma | 1.9 × 10⁻⁴ | mTOR over- |
| | | | | expressing |
| U-118 MG | Central | Glioblastoma | 1.9 × 10⁻⁴ | mTOR |
| | Nervous | | | mutation/ |
| | System (CNS) | | | missense |
| AsPC-1 | Pancreas | Pancreatic | 5.2 × 10⁻⁴ | rictor over- |
| | | | | expressing |
| SCaBER | Bladder | Bladder | 9.5 × 10⁻⁵ | rictor over- |
| | | | | expressing |
| U-138 MG | CNS | Glioblastoma | 9.7 × 10⁻⁵ | raptor over- |
| | | | | expressing, |
| | | | | potentially |
| | | | | non- |
| | | | | tumorigenic |

### Example 2

This example illustrates one method for determining the pharmacokinetics and bioavailability of the compound of Formula I.

A person of skill in the art will be able to determine the pharmacokinetics and bioavailability of the compound of Formula I using in vivo and in vitro methods known to a person of skill in the art, including but not limited to those described below and in Gallant-Haidner et al, 2000 and Trepanier et al, 1998 and references therein. The bioavailability of a compound is determined by a number of factors, (e.g. water solubility, cell membrane permeability, the extent of protein binding and metabolism and stability) each of which may be determined by in vitro tests as described in the examples herein, it will be appreciated by a person of skill in the art that an improvement in one or more of these factors will lead to an improvement in the bioavailability of a compound. Alternatively, the bioavailability of the compound of Formula I may be measured using in vivo methods as described in more detail below, or in the examples herein.

In order to measure bioavailability in vivo, a compound may be administered to a test animal (e.g. mouse or rat) both intraperitoneally (i.p.) or intravenously (i.v.) and orally (p.o.) and blood samples are taken at regular intervals to examine how the plasma concentration of the drug varies over time. The time course of plasma concentration over time can be used to calculate the absolute bioavailability of the compound as a percentage using standard models. An example of a typical protocol is described below.

For example, mice or rats are dosed with 1 or 3 mg/kg of the compound of Formula I i.v. or 1, 5 or 10 mg/kg of the compound of Formula I p.o. Blood samples are taken at 5 min, 15 min, 1 h, 4 h and 24 h intervals, and the concentration of the compound of Formula I in the sample is determined via LCMS-MS. The time-course of plasma or whole blood concentrations can then be used to derive key parameters such as the area under the plasma or blood concentration-time curve (AUC—which is directly proportional to the total amount of unchanged drug that reaches the systemic circulation), the maximum (peak) plasma or blood drug concentration, the time at which maximum plasma or blood drug concentration occurs (peak time), additional factors which are used in the accurate determination of bioavailability include: the compound's terminal half-life, total body clearance, steady-state volume of distribution and F %. These parameters are then analyzed by non-compartmental or compartmental methods to give a calculated percentage bioavailability, for an example of this type of method see Gallant-Haidner et al, 2000 and Trepanier et al, 1998, and references therein.

Shown below are whole blood concentrations following administration of the compound of Formula I to mice. The compound of Formula I was administered to mice at 2 mg/kg or 10 mg/kg and the concentration of the compound in whole blood determined. These determinations were made using protein precipitation, liquid chromatography (LC), and mass spectrometry (MS/MS). Ten μl aliquots of whole blood and matrix calibration standards were distributed in a 96-well plate; 10 μl aliquots of blank matrix for matrix blanks and control blanks were included as controls. Ten μl of water was added to each sample followed by vortexing. One hundred sixty ml of internal standard was added to each sample except the matrix blanks; 160 μl 70:30 water:acetonitrile (ACN) was added to matrix blanks. This was followed by a five-minute vortex at >3500 rpm. One hundred fifty μl of the resultant supernatant was then transferred to a new 96-well plate and the samples blown to dryness at 35° C. The resultant product was then reconstituted with 90 μl ACN. LC was carried out using the equipment, conditions and calibration standards are shown in Tables 5 and 6.

TABLE 5

| | | Time | % | Flow |
|---|---|---|---|---|
| | | (sec) | MPB | (mL/min) |
| LC | | | | |
| Conditions: | | | | |
| Column Id. & | Waters HSS T3, | | | |
| Dimensions: | 30 × 2.1 mm | | | |
| Temperature | Ambient | 15 | 65 | 0.800 |
| (° C.) | | | | |
| Mobile | 0.1% Formic | 75 | 85 | 0.800 |
| Phase A | Acid in | | | |
| | Water | | | |
| Mobile | 0.1% Formic | 5 | 95 | 0.800 |
| Phase B | Acid in | | | |
| | Acetonitrile | | | |
| Needle | 25:25:25:25:0.1 | 25 | 95 | 0.800 |
| Rinse 1 | MeOH:H2O:ACN: | | | |
| | IPA:NH4OH | | | |
| Needle | 10:90:0.1 | 30 | 65 | 0.800 |
| Rinse 2 | MeOH:H2O:FA | | | |

| | |
|---|---|
| MS | |
| Conditions | |
| MS/MS: | API-5500 |
| Ionization | Electrospray |
| Method: | |
| Positive/ | Positive |
| Negative Ion: | |
| Resolution: | Unit |
| Source | 550 |
| Temperature | |
| (° C.): | |
| Transitions | Formula I: 918.5/409.3 Da//Int |
| (m/z): | Std d4-AEA: 352.1/66.0 Da |

TABLE 6
Summary of Calibration Standard Back-Calculated
Concentrations of Formula I in Mouse Whole Blood

| | STD | | | | | | | | | | |
|---|---|---|---|---|---|---|---|---|---|---|---|
| | 1 | 2 | 3 | 4 | 5 | 6 | 7 | 8 | 9 | 10 | 11 |
| | Nominal Concentration (ng/mL) | | | | | | | | | | |
| | 1.00 | 2.00 | 5.00 | 10.0 | 25.0 | 50.0 | 100 | 200 | 500 | 800 | 1000 |
| | NR | NR | NR | 12.4 | 21.0 | 47.6 | 115 | 237 | 580 | 795 | 993 |
| | NR | NR | NR | 9.21 | 20.4 | 40.4 | 95.7 | 208 | 484 | 757 | 978 |
| Mean | NA | NA | NA | 10.8 | 20.7 | 44.0 | 105.4 | 223 | 532 | 776 | 986 |
| SD | NA | NA | NA | 2.26 | 0.424 | 5.09 | 13.6 | 20.5 | 67.9 | 26.9 | 10.61 |
| % Bias | NA | NA | NA | 8.1 | −17.2 | −12.0 | 5.3 | 11.3 | 6.4 | −3.0 | −1.5 |
| n | 0 | 0 | 0 | 2 | 2 | 2 | 2 | 2 | 2 | 2 | 2 |

N/A = Not Applicable//NR = Not Reported

The results of these analyses are summarized in Tables 7 and 8.

TABLE 7

| | Formula I Concentrations (ng/mL) in Mouse Whole Blood | | | | | |
|---|---|---|---|---|---|---|
| Formula 1 (2 mg/kg) | Animal ID | | | | | |
| Time Points (hrs) | 1 | 2 | 3 | Mean | SD | % CV |
| IP (QD × 3 days)- Day 1 | | | | | | |
| 1.00 | 519 | 530 | 537 | 529 | 9.07 | 1.7% |
| 2.00 | 668 | 392 | 233 | 431 | 220 | 51.1% |
| 6.00 | 468 | 646 | 344 | 486 | 152 | 31.2% |
| 24.0 | 35.8 | 48.0 | 26.0 | 36.6 | 11.0 | 30.1% |
| Day 2 | | | | | | |
| 1.00 | 682 | 670 | 354 | 569 | 186 | 32.7% |
| 2.00 | 746 | 841 | 854 | 814 | 59.0 | 7.2% |
| 6.00 | 537 | 803 | 315 | 552 | 244 | 44.3% |
| 24.0 | 26.8 | 76.5 | 40.1 | 47.8 | 25.7 | 53.8% |
| Day 3 | | | | | | |
| 1.00 | 968 | 885 | 986 | 946 | 53.9 | 5.7% |
| 2.00 | 760 | 707 | 282 | 583 | 262 | 44.9% |
| 6.00 | 560 | 860 | 469 | 630 | 205 | 32.5% |
| 24.0 | 45.7 | 92.2 | 49.7 | 62.5 | 25.8 | 41.2% |

TABLE 8

| | Formula I Concentrations (ng/mL) in Mouse Whole Blood | | | | | |
|---|---|---|---|---|---|---|
| Formula 1 (10 mg/kg) | Animal ID | | | | | |
| Time Points (hrs) | 4 | 5 | 6 | Mean | SD | % CV |
| IP (QD × 3 days)- Day 1 | | | | | | |
| 1.00 | 744 | 736 | 853 | 778 | 65.4 | 8.4% |
| 2.00 | 2390 | 2040 | 2590 | 2340 | 278 | 11.9% |
| 6.00 | 1970 | 2300 | 908 | 1726 | 727 | 42.1% |
| 24.0 | 357 | 320 | 141 | 273 | 116 | 42.4% |
| Day 2 | | | | | | |
| 1.00 | AQL | 3130 | 739 | 1935 | NA | NA |
| 2.00 | 3060 | 2660 | 2740 | 2820 | 211.7 | 7.5% |
| 6.00 | AQL | AQL | 811 | 811 | NA | NA |
| 24.0 | 335 | 472 | 199 | 335 | 137 | 40.7% |
| Day 3 | | | | | | |
| 1.00 | 2890 | 3490 | 3220 | 3200 | 300 | 9.4% |
| 2.00 | 2530 | 2680 | 482 | 1897 | 1228 | 64.7% |
| 6.00 | 966 | 2410 | 773 | 1383 | 895 | 64.7% |
| 24.0 | 292 | 548 | 276 | 372 | 153 | 41.0% |

AQL: Original value above quantitation limit (1000 ng/mL). Sample was diluted prior to re-analysis, but due to a sequence error the samples will be re-injected.

As shown above, Formula I exhibits sufficient concentrations in whole blood over time following administration at 2 mg/kg or 10 mg/kg via the intraperitoneal route (IP) once daily (QD) for three days.

### Example 3

### Xenograft Studies

A study was conducted to determine the anti-tumor efficacy of the compound of Formula I on U-118 MG (ATCC® HTB-15, human brain glioblastoma) solid tumors in female nude mice. In this study, advanced-stage subcutaneous xenografts were established to evaluate the antitumor activity of test agents so that clinically relevant parameters of activity could be determined. The end point used to assess drug efficacy was relative tumor growth (comparing tumors in treated versus control mice). In these models, tumor growth were monitored and test agent treatment is typically initiated once tumors reach a weight range of 100-300 mg. Tumor size and body weights were obtained two times per week for determination of toxicity and efficacy. The U-118 MG (ATCC® HTB-15) cell line used in this study was isolated from a malignant glioblastoma taken from a 50-year-old male Caucasian. Study endpoints were determined using the parameters: percent tumor growth inhibition (% TGI)=100 (Wc−Wt)/Wc=100(1−Wt/Wc), where Wc is the Median tumor weight of control group and Wt is the Median tumor weight of the treated group; tumor remission and regression (% REG=100(W0−Wi)W0, where W0 is the Median tumor weight for treated group at the initiation of treatment and Wi is the Median tumor weight for that group at any given time; log₁₀ cell kill=[T−C value in days/(3.32)(Td)], where T−C is the tumor growth delay; the T=Median time (in days) required for the treatment group tumors to reach a predetermined size (i.e., 1,000 mg) and C is the Median time (in days) for the control group to reach the same size; and, tumor-free survivors are excluded from these calculations, where Td=the median tumor doubling time (in days) for the control group.

Female, athymic nude-Foxn1ₙᵤ mice (5-6 weeks old weighing approximately 19-23 grams (mean approx. 21 g) at study initiation (Day 1) (Envigo, Indianapolis, Ind.)) were identified by tail tattoo and housed separately (5 per cage) in Optimax polycarbonate cages with polycarbonate tops, irradiated corn cob bedding, and suspended food and water bottles. During the acclimation and study periods, animals were housed in a laboratory environment with temperatures ranging 67-76° F. and relative humidity of 30%-70%. Automatic timers provided 12 hours of light and 12 hours of dark. Animals were allowed access ad libitum to sterile Harlan Teklad Rodent Chow and sterile, pH 3.0 water. U-118 MG (ATCC® HTB-15) tumor cells were grown in tissue culture and expanded to implant 3×10⁶ cells subcutaneously (SC) in serum-free growth medium on the flank of the mice. Tumor growth was monitored daily. When calculated tumor volume reached approximately 100-300 mm³ (or 100-300 mg), tumor-bearing mice were weighed and randomized into treatment groups. Treatment was initiated after randomization (Study Day 1) and continued as indicated (10 mice per group, QD, 10 ml/kg, either Vehicle Control or Formula I at 10 mg/kg (as a suspension in 2% ethanol, 40% polyethylene glycol 400 (PEG 400), and 58% saline (prepared by dissolving the compound first in 2% ethanol, then adding PEG and saline); suspension prepared every two weeks and frozen at −20° C. between uses). Tumor growth and body weight was measured twice weekly, and animals were observed daily for signs of toxicity and tumor ulceration. Tumor measurements were taken along the length and width using vernier calipers, and tumor volumes were calculated using the following formula: (L×W²)/2. Tumor volume (absolute and percent of baseline) and body weight measurements were compared to vehicle controls using a one-way analysis of variance (ANOVA) with a Dunnett's multiple comparison post-hoc analysis. Significance was set at p≤0.05. Blood samples were collected on Days 16 and 30 from animals 1-5 in Groups 2-4. Whole blood (K₂EDTA, 50 μl/mouse) was collected pre-dose on Day 16 and 24 hours post-dose on Day 30 (trough levels) via retro-orbital blood draw and stored frozen at −80° C. Animals were kept alive after the scheduled study termination date (30 days post treatment initiation) to continue weighing and tumor measurements for evaluation of a possible vehicle effect on tumor growth.

Mice with subcutaneously (SC) implanted tumors were dosed daily (QD) on Days 1-29 by the intraperitoneal (IP) route with vehicle (2% ethanol (EtOH (Sigma))/40% PEG 400 (Sigma)/58% saline (VetPath)) or the compound of Formula I in vehicle (10 mg/kg). Tumor growth and body weights were measured twice weekly as described above, and animals were observed daily for signs of toxicity and tumor ulceration. Efficacy evaluation was based on disease progression after treatment (durable cures), tumor volume, and body weight measurements. As summarized in Table 9, there were no significant differences in body weight following administration of either vehicle control or a compound of Formula I after 30 days.

TABLE 9

| | Day 30 | | |
|---|---|---|---|
| | | Body Wt. % | Change in |
| | Body Weight | Δ from | Body Weight |
| IP, QD | (g) | Baseline | Day 1-30 |
| Group 1 | | | |
| Vehicle | | | |
| Control | | | |
| 1 | 20.44 | 9.89% | 1.84 |
| 2 | 20.48 | 8.47% | 1.60 |
| 3 | 21.22 | 11.80% | 2.24 |
| 4 | 21.48 | 13.05% | 2.48 |
| 5 | 22.02 | 14.87% | 2.85 |
| 6 | 21.80 | 13.07% | 2.52 |
| 7 | 21.47 | 11.01% | 2.13 |
| 8 | 21.28 | 9.63% | 1.87 |
| 9 | 22.32 | 13.88% | 2.72 |
| 10 | 21.97 | 11.92% | 2.34 |
| Mean | 21.45 | 11.76% | 2.26 |
| Median | 21.48 | 11.86% | 2.29 |
| SE | 0.20 | 0.64% | 0.13 |
| Percent | 100% | 100% | |
| Survival | | | |
| Group 2 | | | |
| Formula I | | | |
| 1 | 22.87 | 15.04% | 2.99 |
| 2 | 23.14 | 16.22% | 3.23 |
| 3 | 21.89 | 9.83% | 1.96 |
| 4 | 23.02 | 15.16% | 3.03 |
| 5 | 21.90 | 8.63% | 1.74 |
| 6 | 24.53 | 20.66% | 4.20 |
| 7 | 23.65 | 16.05% | 3.27 |
| 8 | 23.02 | 12.90% | 2.63 |
| 9 | 23.85 | 14.77% | 3.07 |
| 10 | 23.62 | 13.12% | 2.74 |
| Mean | 23.15 | 14.24% | 2.89 |
| Median | 23.08 | 14.91% | 3.01 |
| SE | 0.26 | 1.08% | 0.22 |
| ANOVA | | 0.465 | 0.341 |
| (Dunnett's) vs. | | | |
| Group 1 | | | |
| Percent Survival | 100% | 100% | |

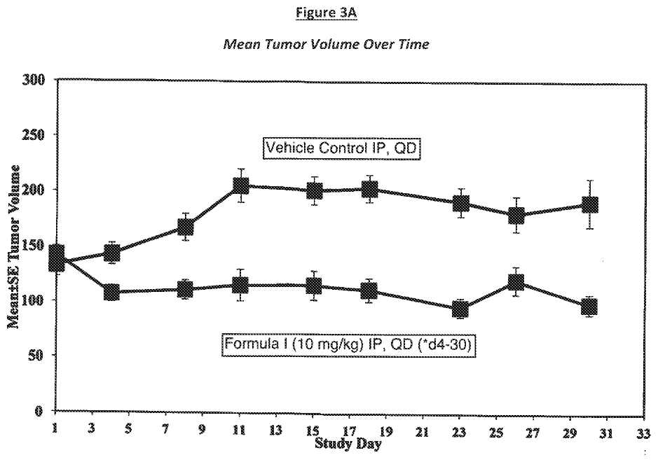

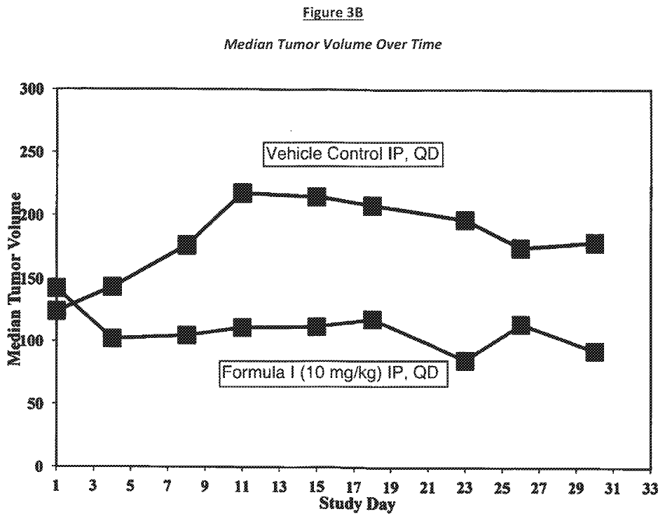

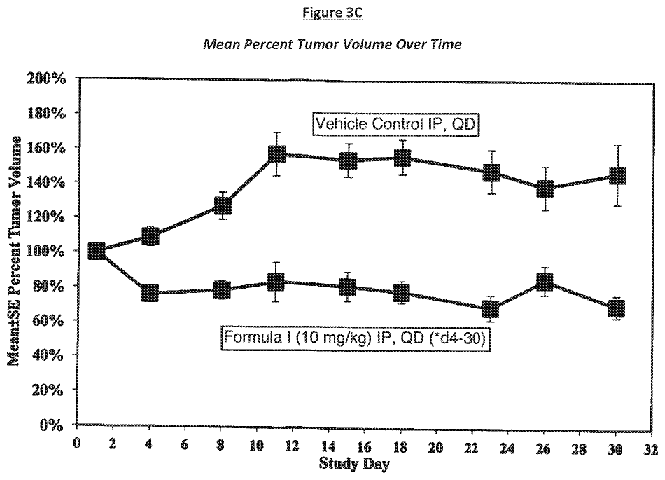

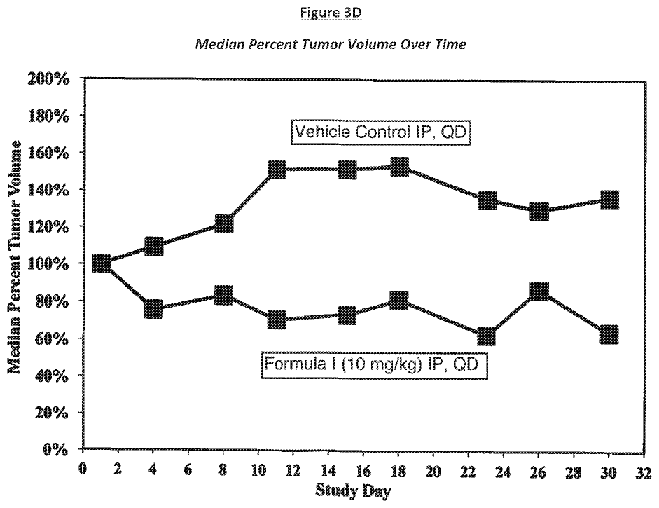

The data presented in Tables 10-16 and FIGS. 3A-D shows that administration of the compound of Formula I at 10 mg/kg was effective in reducing U118 tumor growth.

TABLE 10

| | Day 1 | | | Day 4 | | | |
|---|---|---|---|---|---|---|---|
| | Length | Width | Tumor | Length | Width | Tumor | % Tumor |
| | (mm) | (mm) | Vol. (mm²) | (mm) | (mm) | Vol. (mm³) | Volume |
| Vehicle | | | | | | | |
| Control | | | | | | | |
| IP, QD | | | | | | | |
| 1 | 9.70 | 4.67 | 105.77 | 9.41 | 4.44 | 92.75 | 88% |
| 2 | 9.68 | 6.57 | 208.92 | 10.30 | 6.41 | 211.60 | 101% |
| 3 | 11.81 | 4.47 | 117.99 | 11.56 | 4.98 | 143.35 | 121% |
| 4 | 9.54 | 5.09 | 123.58 | 10.47 | 4.88 | 124.67 | 101% |
| 5 | 7.76 | 5.81 | 130.97 | 9.71 | 5.76 | 161.08 | 123% |
| 6 | 8.35 | 4.95 | 102.30 | 8.96 | 5.31 | 126.32 | 123% |
| 7 | 7.36 | 6.22 | 142.37 | 7.46 | 5.89 | 129.40 | 91% |
| 8 | 7.54 | 5.48 | 113.21 | 7.98 | 6.18 | 152.39 | 135% |
| 9 | 8.31 | 6.29 | 164.39 | 7.95 | 6.01 | 143.58 | 87% |
| 10 | 7.76 | 5.67 | 124.74 | 7.22 | 6.37 | 146.48 | 117% |
| Mean | | | 133.42 | | | 143.16 | 109% |
| Median | | | 124.16 | | | 143.46 | 109% |
| SE | | | 10.16 | | | 9.69 | 5% |
| Formula I | | | | | | | |
| IP, QD | | | | | | | |
| 1 | 8.56 | 5.71 | 139.55 | 7.97 | 4.94 | 97.25 | 70% |
| 2 | 8.03 | 6.29 | 158.85 | 7.23 | 5.70 | 117.45 | 74% |
| 3 | 7.10 | 5.87 | 122.32 | 5.88 | 5.16 | 78.28 | 64% |
| 4 | 6.89 | 5.88 | 119.11 | 7.19 | 5.36 | 103.28 | 87% |
| 5 | 8.14 | 6.18 | 155.44 | 8.57 | 5.31 | 120.82 | 78% |
| 6 | 9.73 | 5.46 | 145.03 | 10.03 | 5.38 | 145.16 | 100% |
| 7 | 7.48 | 5.20 | 101.13 | 7.17 | 5.06 | 91.79 | 91% |
| 8 | 8.29 | 5.60 | 129.99 | 7.34 | 4.61 | 78.00 | 60% |
| 9 | 9.55 | 6.02 | 173.05 | 9.67 | 5.42 | 142.03 | 82% |
| 10 | 8.16 | 6.60 | 177.72 | 7.52 | 5.18 | 100.89 | 57% |
| Mean | | | 142.22 | | | 107.49 | 76% |
| Median | | | 142.29 | | | 102.09 | 76% |
| SE | | | 7.78 | | | 7.47 | 4% |
| ANOVA | | | 0.853 | | | 0.025 | 0.001 |
| (Dunnett's) | | | | | | | |
| vs. Group 1 | | | | | | | |

TABLE 11

| | Day 8 | | | | Day 11 | | | |
|---|---|---|---|---|---|---|---|---|
| | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| Group 1 | | | | | | | | |
| Vehicle | | | | | | | | |
| Control | | | | | | | | |
| 1 | 9.37 | 5.02 | 118.06 | 112% | 11.20 | 4.45 | 110.89 | 105% |
| 2 | 9.51 | 7.08 | 238.35 | 114% | 10.42 | 6.89 | 247.33 | 118% |
| 3 | 11.71 | 5.62 | 184.93 | 157% | 12.40 | 6.31 | 246.86 | 209% |
| 4 | 9.84 | 4.60 | 104.11 | 84% | 9.79 | 5.36 | 140.63 | 114% |
| 5 | 10.13 | 6.06 | 186.01 | 142% | 9.90 | 7.17 | 254.47 | 194% |
| 6 | 10.04 | 5.90 | 174.75 | 171% | 9.56 | 6.84 | 223.64 | 219% |
| 7 | 9.04 | 6.28 | 178.26 | 125% | 9.06 | 6.82 | 210.70 | 148% |
| 8 | 8.92 | 5.49 | 134.42 | 119% | 8.46 | 6.45 | 175.98 | 155% |
| 9 | 7.69 | 6.87 | 181.47 | 110% | 9.05 | 7.16 | 231.98 | 141% |
| 10 | 7.51 | 6.73 | 170.07 | 136% | 8.28 | 7.15 | 211.65 | 170% |
| Mean | | | 167.04 | 127% | | | 205.41 | 157% |
| Median | | | 176.50 | 122% | | | 217.64 | 152% |
| SE | | | 12.29 | 8% | | | 15.27 | 13% |
| Group 2 | | | | | | | | |
| Formula I | | | | | | | | |
| IP, QD | | | | | | | | |
| 1 | 7.52 | 4.78 | 85.91 | 62% | 7.62 | 3.87 | 57.06 | 41% |
| 2 | 7.13 | 5.50 | 107.84 | 68% | 6.93 | 5.63 | 109.83 | 69% |
| 3 | 6.46 | 5.70 | 104.94 | 86% | 6.45 | 4.96 | 79.34 | 65% |
| 4 | 7.64 | 5.07 | 98.19 | 82% | 7.23 | 6.01 | 130.57 | 110% |
| 5 | 7.95 | 6.40 | 162.82 | 105% | 8.03 | 5.29 | 112.36 | 72% |
| 6 | 8.95 | 5.33 | 127.13 | 88% | 9.78 | 6.31 | 194.70 | 134% |
| 7 | 7.98 | 4.95 | 97.76 | 97% | 7.88 | 5.95 | 139.49 | 138% |
| 8 | 7.27 | 4.50 | 73.61 | 57% | 8.35 | 4.52 | 85.30 | 66% |
| 9 | 9.79 | 5.47 | 146.46 | 85% | 9.55 | 6.07 | 175.93 | 102% |
| 10 | 6.89 | 5.51 | 104.59 | 59% | 6.39 | 4.72 | 71.18 | 40% |
| Mean | | | 110.93 | 79% | | | 115.58 | 84% |
| Median | | | 104.77 | 84% | | | 111.09 | 71% |
| SE | | | 8.60 | 5% | | | 14.29 | 11% |
| ANOVA | | | 0.003 | 0.000 | | | 0.000 | 0.001 |
| (Dunnett's) | | | | | | | | |
| vs. Group 1 | | | | | | | | |

TABLE 12

| | Day 15 | | | | Day 18 | | | |
|---|---|---|---|---|---|---|---|---|
| | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| Group 1 | | | | | | | | |
| Vehicle | | | | | | | | |
| Control | | | | | | | | |
| 1 | 10.01 | 5.09 | 129.67 | 123% | 12.20 | 4.99 | 151.89 | 144% |
| 2 | 10.95 | 6.55 | 234.89 | 112% | 10.08 | 7.01 | 247.67 | 119% |
| 3 | 12.39 | 6.25 | 241.99 | 205% | 10.65 | 6.29 | 210.68 | 179% |
| 4 | 10.00 | 5.58 | 155.68 | 126% | 10.49 | 5.40 | 152.94 | 124% |
| 5 | 11.55 | 6.34 | 232.13 | 177% | 12.11 | 6.46 | 252.68 | 193% |
| 6 | 9.60 | 5.81 | 162.03 | 158% | 9.62 | 5.90 | 167.44 | 164% |
| 7 | 9.67 | 6.26 | 189.47 | 133% | 9.35 | 6.17 | 177.97 | 125% |
| 8 | 8.81 | 6.83 | 205.49 | 182% | 9.38 | 7.19 | 242.45 | 214% |
| 9 | 9.86 | 6.96 | 238.82 | 145% | 9.46 | 6.88 | 223.89 | 136% |
| 10 | 8.56 | 7.24 | 224.35 | 180% | 8.54 | 6.94 | 205.66 | 165% |
| Mean | | | 201.45 | 154% | | | 203.33 | 156% |
| Median | | | 214.92 | 152% | | | 208.17 | 154% |
| SE | | | 12.73 | 10% | | | 12.26 | 10% |
| Group 2 | | | | | | | | |
| Formula I | | | | | | | | |
| IP, QD | | | | | | | | |
| 1 | 7.71 | 4.66 | 83.71 | 60% | 7.52 | 4.28 | 68.88 | 49% |
| 2 | 7.63 | 5.62 | 120.49 | 76% | 7.91 | 5.65 | 126.25 | 79% |
| 3 | 5.85 | 4.62 | 62.43 | 51% | 5.30 | 4.69 | 58.29 | 48% |
| 4 | 7.46 | 4.37 | 71.23 | 60% | 8.07 | 5.05 | 102.90 | 86% |
| 5 | 8.23 | 5.18 | 110.42 | 71% | 8.12 | 5.66 | 130.06 | 84% |
| 6 | 10.03 | 6.35 | 202.22 | 139% | 9.56 | 5.65 | 152.59 | 105% |
| 7 | 8.31 | 5.10 | 108.07 | 107% | 6.33 | 4.93 | 76.93 | 76% |
| 8 | 8.98 | 5.14 | 118.62 | 91% | 7.91 | 5.83 | 134.43 | 103% |
| 9 | 8.27 | 5.24 | 113.54 | 66% | 8.63 | 6.01 | 155.86 | 90% |
| 10 | 8.14 | 6.41 | 167.23 | 94% | 6.45 | 5.85 | 110.37 | 62% |
| Mean | | | 115.80 | 81% | | | 111.66 | 78% |
| Median | | | 111.98 | 73% | | | 118.31 | 82% |
| SE | | | 13.38 | 9% | | | 10.88 | 6% |
| ANOVA | | | 0.000 | 0.000 | | | 0.000 | 0.000 |
| (Dunnett's) | | | | | | | | |
| vs. Group 1 | | | | | | | | |

TABLE 13

| | Day 23 | | | | Day 26 | | | |
|---|---|---|---|---|---|---|---|---|
| | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| Group 1 | | | | | | | | |
| Vehicle | | | | | | | | |
| Control | | | | | | | | |
| 1 | 12.08 | 4.86 | 142.66 | 135% | 11.80 | 5.68 | 190.35 | 180% |
| 2 | 11.02 | 6.65 | 243.67 | 117% | 12.12 | 6.75 | 276.11 | 132% |
| 3 | 10.06 | 6.57 | 217.12 | 184% | 11.34 | 5.16 | 150.97 | 128% |
| 4 | 9.91 | 5.09 | 128.37 | 104% | 8.40 | 4.98 | 104.16 | 84% |
| 5 | 12.54 | 6.04 | 228.74 | 175% | 11.69 | 6.36 | 236.43 | 181% |
| 6 | 9.68 | 6.41 | 198.87 | 194% | 10.21 | 6.33 | 204.55 | 200% |
| 7 | 9.80 | 6.30 | 194.48 | 137% | 9.97 | 5.84 | 170.02 | 119% |
| 8 | 9.71 | 7.00 | 237.90 | 210% | 9.94 | 6.01 | 179.52 | 159% |
| 9 | 7.74 | 6.76 | 176.85 | 108% | 8.20 | 6.36 | 165.84 | 101% |
| 10 | 7.51 | 6.24 | 146.21 | 117% | 6.68 | 6.24 | 130.05 | 104% |
| Mean | | | 191.49 | 148% | | | 180.80 | 139% |
| Median | | | 196.67 | 136% | | | 174.77 | 130% |
| SE | | | 13.17 | 12% | | | 15.82 | 12% |
| Group 2 | | | | | | | | |
| Formula I | | | | | | | | |
| IP, QD | | | | | | | | |
| 1 | 7.55 | 4.59 | 79.53 | 57% | 8.09 | 5.44 | 119.71 | 86% |
| 2 | 7.15 | 5.01 | 89.73 | 56% | 6.94 | 4.93 | 84.34 | 53% |
| 3 | 6.11 | 4.57 | 63.80 | 52% | 5.84 | 4.67 | 63.68 | 52% |
| 4 | 8.31 | 5.77 | 138.33 | 116% | 7.18 | 4.81 | 83.06 | 70% |
| 5 | 6.97 | 4.42 | 68.08 | 44% | 8.54 | 5.66 | 136.79 | 88% |
| 6 | 9.51 | 5.09 | 123.19 | 85% | 9.85 | 6.22 | 190.54 | 131% |
| 7 | 6.19 | 5.11 | 80.82 | 80% | 7.60 | 5.35 | 108.77 | 108% |
| 8 | 7.90 | 5.51 | 119.92 | 92% | 8.61 | 5.57 | 133.56 | 103% |
| 9 | 7.29 | 4.53 | 74.80 | 43% | 9.46 | 4.79 | 108.53 | 63% |
| 10 | 6.60 | 6.08 | 121.99 | 69% | 7.26 | 7.04 | 179.91 | 101% |
| Mean | | | 96.02 | 69% | | | 120.89 | 85% |
| Median | | | 85.27 | 63% | | | 114.24 | 87% |
| SE | | | 8.55 | 7% | | | 12.95 | 8% |
| ANOVA | | | 0.000 | 0.000 | | | 0.006 | 0.002 |
| (Dunnett's) | | | | | | | | |
| vs. Group 1 | | | | | | | | |

TABLE 14

Day 30

| | Length | Width | Tumor | % Tumor |
|---|---|---|---|---|
| IP, QD | (mm) | (mm) | Vol. (mm³) | Volume |
| Group 1 | | | | |
| Vehicle | | | | |
| Control | | | | |
| 1 | 11.06 | 5.19 | 148.96 | 141% |
| 2 | 10.56 | 6.43 | 218.30 | 104% |
| 3 | 9.37 | 5.66 | 150.09 | 127% |
| 4 | 6.89 | 4.87 | 81.70 | 66% |
| 5 | 11.14 | 7.53 | 315.82 | 241% |
| 6 | 10.16 | 5.77 | 169.13 | 165% |
| 7 | 9.78 | 6.21 | 188.58 | 132% |
| 8 | 9.75 | 7.42 | 268.40 | 237% |
| 9 | 9.48 | 7.01 | 232.92 | 142% |
| 10 | 8.27 | 5.80 | 139.10 | 112% |
| Mean | | | 191.30 | 147% |
| Median | | | 178.85 | 137% |
| SE | | | 21.77 | 18% |
| Group 2 | | | | |
| Formula I | | | | |
| 1 | 7.42 | 4.87 | 87.99 | 63% |
| 2 | 7.01 | 5.28 | 97.71 | 62% |
| 3 | 6.91 | 4.34 | 65.08 | 53% |
| 4 | 7.82 | 5.45 | 116.14 | 98% |
| 5 | 6.80 | 4.96 | 83.65 | 54% |
| 6 | 9.97 | 5.69 | 161.39 | 111% |
| 7 | 8.07 | 4.49 | 81.35 | 80% |
| 8 | 6.63 | 4.46 | 65.94 | 51% |
| 9 | 8.02 | 5.31 | 113.07 | 65% |
| 10 | 7.52 | 5.68 | 121.31 | 68% |
| Mean | | | 99.36 | 71% |
| Median | | | 92.85 | 64% |
| SE | | | 9.31 | 6% |
| ANOVA | | | 0.000 | 0.000 |
| (Dunnett's) | | | | |
| vs. Group 1 | | | | |

TABLE 15
Summary of Statistical Analysis (Tumor Volume)
Tumor Volume (Day 1)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | −8.795 | −41.96 to | No | ns | 0.8532 | | | |
| | | 24.37 | | | | | | |
| Test details | Mean 1 | Mean 2 | Mean Diff. | SE of diff. | n1 | n2 | q | DF |
| Vehicle Control vs. Formula I | 133.4 | 142.2 | −8.795 | 13.53 | 10 | 10 | 0.6502 | 36 |

Tumor Volume (Day 4)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 35.67 | 3.896 to | Yes | * | 0.0247 | | | |
| | | 67.44 | | | | | | |
| Test details | Mean 1 | Mean 2 | Mean Diff. | SE of diff. | n1 | n2 | q | DF |
| Vehicle Control vs. Formula I | 143.2 | 107.5 | 35.67 | 12.96 | 10 | 10 | 2.753 | 36 |

Tumor Volume (Day 8)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 56.12 | 18.13 to | Yes | ** | 0.0025 | | | |
| | | 94.1 | | | | | | |
| Test details | Mean 1 | Mean 2 | Mean Diff. | SE of diff. | n1 | n2 | q | DF |
| Vehicle Control vs. Formula I | 167 | 110.9 | 56.12 | 15.49 | 10 | 10 | 3.623 | 36 |

Tumor Volume (Day 11)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 89.84 | 41.11 to | Yes | *** | 0.0002 | | | |
| | | 138.6 | | | | | | |
| Test details | Mean 1 | Mean 2 | Mean Diff. | SE of diff. | n1 | n2 | q | DF |
| Vehicle Control vs. Formula I | 205.4 | 115.6 | 89.84 | 19.87 | 10 | 10 | 4.521 | 36 |

Tumor Volume (Day 15)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 85.66 | 44.69 to | Yes | **** | 0.0001 | | | |
| | | 126.6 | | | | | | |
| Test details | Mean 1 | Mean 2 | Mean Diff. | SE of diff. | n1 | n2 | q | DF |
| Vehicle Control vs. Formula I | 201.5 | 115.8 | 85.66 | 16.7 | 10 | 10 | 5.128 | 36 |

Tumor Volume (Day 18)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 91.67 | 54.95 to | Yes | **** | 0.0001 | | | |
| | | 128.4 | | | | | | |
| Test details | Mean 1 | Mean 2 | Mean Diff. | SE of diff. | n1 | n2 | q | DF |
| Vehicle Control vs. Formula I | 203.3 | 111.7 | 91.67 | 14.97 | 10 | 10 | 6.122 | 36 |

Tumor Volume (Day 23)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 95.47 | 60.08 to | Yes | | 0.0001 | | | |
| | | 130.9 | | | | | | |
| Test details | Mean 1 | Mean 2 | Mean Diff. | SE of diff. | n1 | n2 | q | DF |
| Vehicle Control vs. Formula I | 191.5 | 96.02 | 95.47 | 14.43 | 10 | 10 | 6.616 | 36 |

Tumor Volume (Day 26)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 59.91 | 15.75 to | Yes | ** | 0.0057 | | | |
| | | 104.1 | | | | | | |
| Test details | Mean 1 | Mean 2 | Mean Diff. | SE of diff. | n1 | n2 | q | DF |
| Vehicle Control vs. Formula I | 180.8 | 120.9 | 59.91 | 18.01 | 10 | 10 | 3.327 | 36 |

Tumor Volume (Day 30)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 91.94 | 43.74 to | Yes | *** | 0.0001 | | | |
| | | 140.1 | | | | | | |
| Test details | Mean 1 | Mean 2 | Mean Diff. | SE of diff. | n1 | n2 | q | DF |
| Vehicle Control vs. Formula I | 191.3 | 99.36 | 91.94 | 19.65 | 10 | 10 | 4.678 | 36 |

TABLE 16
Summary of Statistical Analysis (Tumor Percent)
Tumor Percent (Day 4)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 0.3263 | 0.1229 to | Yes | ** | 0.0011 | | | |
| | | 0.5298 | | | | | | |
| Test details | Mean 1 | Mean 2 | Mean Diff. | SE of diff. | n1 | n2 | q | DF |
| Vehicle Control vs. Formula I | 1.088 | 0.7618 | 0.3263 | 0.08296 | 10 | 10 | 3.933 | 36 |

Tumor Percent (Day 8)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 0.4833 | 0.232 to | Yes | *** | 0.0001 | | | |
| | | 0.7346 | | | | | | |
| Test details | Mean 1 | Mean 2 | Mean Diff. | SE of diff. | n1 | n2 | q | DF |
| Vehicle Control vs. Formula I | 1.27 | 0.7869 | 0.4833 | 0.1025 | 10 | 10 | 4.717 | 36 |

Tumor Percent (Day 11)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 0.7371 | 0.303 to | Yes | *** | 0.0006 | | | |
| | | 1.171 | | | | | | |
| Test details | Mean 1 | Mean 2 | Mean Diff. | SE of diff. | n1 | n2 | q | DF |
| Vehicle Control vs. Formula I | 1.573 | 0.8363 | 0.7371 | 0.177 | 10 | 10 | 4.165 | 36 |

Tumor Percent (Day 15)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 0.7265 | 0.4312 to | Yes | **** | 0.0001 | | | |
| | | 1.022 | | | | | | |
| Test details | Mean 1 | Mean 2 | Mean Diff. | SE of diff. | n1 | n2 | q | DF |
| Vehicle Control vs. Formula I | 1.541 | 0.815 | 0.7265 | 0.1204 | 10 | 10 | 6.033 | 36 |

Tumor Percent (Day 18)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 0.7779 | 0.4569 to | Yes | **** | 0.0001 | | | |
| | | 1.099 | | | | | | |
| Test details | Mean 1 | Mean 2 | Mean Diff. | SE of diff. | n1 | n2 | q | DF |
| Vehicle Control vs. Formula I | 1.561 | 0.7834 | 0.7779 | 0.1309 | 10 | 10 | 5.943 | 36 |

Tumor Percent (Day 23)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 0.7854 | 0.4641 to | Yes | **** | 0.0001 | | | |
| | | 1.107 | | | | | | |
| Test details | Mean 1 | Mean 2 | Mean Diff. | SE of diff. | n1 | n2 | q | DF |
| Vehicle Control vs. Formula I | 1.48 | 0.6946 | 0.7854 | 0.131 | 10 | 10 | 5.995 | 36 |

Tumor Percent (Day 26)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 0.5337 | 0.1908 to | Yes | ** | 0.0015 | | | |
| | | 0.8765 | | | | | | |
| Test details | Mean 1 | Mean 2 | Mean Diff. | SE of diff. | n1 | n2 | q | DF |
| Vehicle Control vs. Formula I | 1.388 | 0.8543 | 0.5337 | 0.1398 | 10 | 10 | 3.817 | 36 |

Tumor Percent (Day 30)

| Dunnett's multiple comparisons | Mean | 95.00% CI | | | | | | |
|---|---|---|---|---|---|---|---|---|
| test | Diff. | of diff. | Significant? | Summary | Adjusted P Value | | | |
| Vehicle Control vs. Formula I | 0.7627 | 0.3817 to | Yes | **** | 0.0001 | | | |
| | | 1.144 | | | | | | |
| Test details | Mean 1 | Mean 2 | Mean Diff. | SE of diff. | n1 | n2 | q | DF |
| Vehicle Control vs. Formula I | 1.468 | 0.7051 | 0.7627 | 0.1554 | 10 | 10 | 4.91 | 36 |

As shown in Tables 10-16 and FIGS. 3A-3D, the compound of Formula I significantly reduced tumor growth in the U188 xenograft model over the 30-day test period and showed effects as early as four days following administration of Formula I (10 mg/kg, intraperitoneal (IP) administration once per day (QD)) as compared to administration of the Vehicle Control. Thus, treatment with the compound of Formula I showed significant inhibitory effect on U-118 MG solid tumor growth in female nude mice as determined by evaluation of tumor size overtime. Survival for animals treated with Vehicle Control or Formula I was 100% at 30 days.

While certain embodiments have been described in terms of the preferred embodiments, it is understood that variations and modifications will occur to those skilled in the art. Therefore, it is intended that the appended claims cover all such equivalent variations that come within the scope of the following claims.

What is claimed is:

1. A method of preventing and/or treating cancer in a mammal, the method comprising administering to said mammal an effective amount of a compound of Formula I: 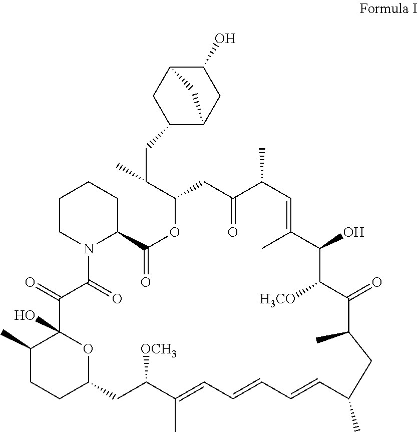 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2C[C@@H]3C[C@H]2C[C@H]3O)CC(=O)[C@H](C)/C=C(\C)[C@@H](O)[C@@H](OC)C(=O)[C@H](C)C[C@H](C)/C=C/C=C/C=C/1C]
or a pharmaceutically acceptable salt thereof, solvate thereof, ester thereof, or mixture thereof and/or comprising the compound of Formula I; and/or a composition comprising Formula I.

2. The method of claim 1, wherein the mammal is human.

3. The method of claim 1 or 2, wherein the cancer is selected from the group consisting of a blood cancer, bone cancer, solid tumor, adenocarcinoma, brain cancer, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and transitional cell carcinoma.

4. The method of any preceding claim wherein the compound of Formula I is administered as the sole active pharmaceutical agent; or the compound of Formula I is administered in combination with one or more of a chemotherapeutic agent, anti-cancer agent, or immune modulator; and/or radiation therapy and/or surgery.

5. The method of any preceding claim wherein the administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately.

6. The method of any preceding claim wherein the compound of Formula I is provided as a bead, tablet, capsule, solution, or suspension.

7. Use of a compound of Formula I in the preparation of a medicament for the prevention and/or treatment of cancer.

8. The use of claim 7 wherein the cancer is selected from the group consisting of a blood cancer, bone cancer, solid tumor, adenocarcinoma, brain cancer, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and transitional cell carcinoma.

9. The use of claim 7 or 8 wherein the compound of Formula I is administered as the sole active pharmaceutical agent; or the compound of Formula I is administered in combination with one or more chemotherapeutic agents, anti-cancer agents, radiation therapy, and immune modulators.

10. The use of any one of claims 7-9 wherein the administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately.

11. The use of any one of claims 7-10 wherein the compound of Formula I is provided as a bead, tablet, capsule, solution, or suspension.

12. A kit for preventing and/or treating cancer, the kit comprising at least one therapeutically effective dose of the compound of Formula I, and instructions for preventing and/or treating cancer using the same.

13. The kit of claim 10 wherein the instructions refer to administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately.

14. The kit of claim 12 or 13 wherein the compound of Formula I is provided as a bead, tablet, capsule, solution, solid, or suspension.
